SEQUENCE LISTING (48 sequences)

SEQ ID NO: 1 - protein, 59 aa, Artificial Sequence
  Regulon
HKKWQFNSPFVPRADEPARKGKVHIPFPLDNITCRVPMAREPTVIHGKREVTLHLHPDH

SEQ ID NO: 2 - protein, 14 aa, Artificial Sequence
  Regulon_construct1
PTVIHGKREVTLHL

SEQ ID NO: 3 - protein, 23 aa, Artificial Sequence
  Regulon_construct4
PMAREPTVIHGKREVTLHLHPDH

SEQ ID NO: 4 - protein, 20 aa, Artificial Sequence
  RH_construct1
ELKHFEAKIEKHNHYQKQLE

SEQ ID NO: 5 - protein, 41 aa, Artificial Sequence
  RH_construct2
DKELEAFREELKHFEAKIEKHNHYQKQLEIAHEKLRHAESV

SEQ ID NO: 6 - protein, 84 aa, Artificial Sequence
  RH_construct3
DKELEAFREELKHFEAKIEKHNHYQKQLEIAHEKLRHAESVGDGERVSRSREKHALLEGRTKELGYTVKKHLQDLSGRISRARH

SEQ ID NO: 7 - protein, 19 aa, Artificial Sequence
  Flex_1
TGESNTVRGKRGSYKDENR

SEQ ID NO: 8 - protein, 19 aa, Artificial Sequence
  Flex_2
FRESNTIRGKRETTKDENR

SEQ ID NO: 9 - protein, 19 aa, Artificial Sequence
  Flex_3
TKETSATRGKRETTKDEGK

SEQ ID NO: 10 - protein, 19 aa, Artificial Sequence
  Flex_4
ARETSIVRGKRDYFKDEGK

SEQ ID NO: 11 - protein, 19 aa, Artificial Sequence
  Flex_5
SSESNITRGKREYTKDEGR

SEQ ID NO: 12 - protein, 20 aa, Artificial Sequence
  Rigid_1
GDAAAAKAAKAAAKAAADGY

SEQ ID NO: 13 - protein, 20 aa, Artificial Sequence
  Rigid_2
GDAAAARAAKAAARAAADGY

SEQ ID NO: 14 - protein, 34 aa, Artificial Sequence
  Rigid_3
GDAAAAKAAKAAAKAAAAAAKAAKAAAKAAADGY

SEQ ID NO: 15 - protein, 34 aa, Artificial Sequence
  Rigid_4
GDAAEAKAEKAEAKAEAAEAKAEKAEAKAAAEGY

SEQ ID NO: 16 - protein, 34 aa, Artificial Sequence
  Rigid_5
GDAAEAKAQKAQAKANAAKAKAQKAQAKAAANGY

SEQ ID NO: 17 - protein, 6 aa, Artificial Sequence
  Linker
GSGGGG

SEQ ID NO: 18 - protein, 8 aa, Artificial Sequence
  Linker
GSGSGGGG

SEQ ID NO: 19 - protein, 6 aa, Artificial Sequence
  Linker
ASGGGS

SEQ ID NO: 20 - protein, 12 aa, Artificial Sequence
  Regulon hairpin
TVIHGKREVTLH

SEQ ID NO: 21 - protein, 5 aa, Artificial Sequence
  Beta strand
TVIHG

SEQ ID NO: 22 - protein, 5 aa, Artificial Sequence
  Beta strand
EVTLH

SEQ ID NO: 23 - protein, 19 aa, Artificial Sequence
  Consensus sequence
XXEXXXXRGKRXXXKDEXX

SEQ ID NO: 24 - protein, 10 aa, Artificial Sequence
  Consensus sequence
RGKRXXXKDE

SEQ ID NO: 25 - protein, 18 aa, Artificial Sequence
  Consensus sequence
XEXXXXRGKRXXXKDEXX

SEQ ID NO: 26 - protein, 17 aa, Artificial Sequence
  Consensus sequence
EXXXXRGKRXXXKDEXX

SEQ ID NO: 27 - protein, 16 aa, Artificial Sequence
  Consensus sequence
XXXXRGKRXXXKDEXX

SEQ ID NO: 28 - protein, 15 aa, Artificial Sequence
  Consensus sequence
XXXRGKRXXXKDEXX

SEQ ID NO: 29 - protein, 14 aa, Artificial Sequence
  Consensus sequence
XXRGKRXXXKDEXX

SEQ ID NO: 30 - protein, 13 aa, Artificial Sequence
  Consensus sequence
XRGKRXXXKDEXX

SEQ ID NO: 31 - protein, 12 aa, Artificial Sequence
  Consensus sequence
RGKRXXXKDEXX

SEQ ID NO: 32 - protein, 18 aa, Artificial Sequence
  Consensus sequence
XXEXXXXRGKRXXXKDEX

SEQ ID NO: 33 - protein, 17 aa, Artificial Sequence
  Consensus sequence
XEXXXXRGKRXXXKDEX

SEQ ID NO: 34 - protein, 16 aa, Artificial Sequence
  Consensus sequence
EXXXXRGKRXXXKDEX

SEQ ID NO: 35 - protein, 15 aa, Artificial Sequence
  Consensus sequence
XXXXRGKRXXXKDEX

SEQ ID NO: 36 - protein, 14 aa, Artificial Sequence
  Consensus sequence
XXXRGKRXXXKDEX

SEQ ID NO: 37 - protein, 13 aa, Artificial Sequence
  Consensus sequence
XXRGKRXXXKDEX

SEQ ID NO: 38 - protein, 12 aa, Artificial Sequence
  Consensus sequence
XRGKRXXXKDEX

SEQ ID NO: 39 - protein, 11 aa, Artificial Sequence
  Consensus sequence
RGKRXXXKDEX

SEQ ID NO: 40 - protein, 17 aa, Artificial Sequence
  Consensus sequence
XXEXXXXRGKRXXXKDE

SEQ ID NO: 41 - protein, 16 aa, Artificial Sequence
  Consensus sequence
XEXXXXRGKRXXXKDE

SEQ ID NO: 42 - protein, 15 aa, Artificial Sequence
  Consensus sequence
EXXXXRGKRXXXKDE

SEQ ID NO: 43 - protein, 14 aa, Artificial Sequence
  Consensus sequence
XXXXRGKRXXXKDE

SEQ ID NO: 44 - protein, 13 aa, Artificial Sequence
  Consensus sequence
XXXRGKRXXXKDE

SEQ ID NO: 45 - protein, 12 aa, Artificial Sequence
  Consensus sequence
XXRGKRXXXKDE

SEQ ID NO: 46 - protein, 11 aa, Artificial Sequence
  Consensus sequence
XRGKRXXXKDE

SEQ ID NO: 47 - protein, 10 aa, Artificial Sequence
  Consensus sequence
RGKRXXXKDE

SEQ ID NO: 48 - protein, 357 aa, Homo sapiens
MAPRRVRSFLRGLPALLLLLLFLGPWPAASHGGKYSREKNQPKPSPKRESGEEFRMEKLNQLWEKAQRLHLPPVRLAELHADLKIQERDELAWKKLKLDGLDEDGEKEARLIRNLNVILAKYGLDGKKDARQVTSNSLSGTQEDGLDDPRLEKLWHKAKTSGKFSGEELDKLWREFLHHKEKVHEYNVLLETLSRTEEIHENVISPSDLSDIKGSVLHSRHTELKEKLRSINQGLDRLRRVSHQGYSTEAEFEEPRVIDLWDLAQSANLTDKELEAFREELKHFEAKIEKHNHYQKQLEIAHEKLRHAESVGDGERVSRSREKHALLEGRTKELGYTVKKHLQDLSGRISRARHNEL

(19) United States
(12) Patent Application Publication
(10) Pub. No.: US 2015/0376237 A1
(43) Pub. Date: Dec. 31, 2015

(54) POLYPEPTIDES FOR BLOOD BRAIN BARRIER TRANSPORT

(71) Applicant: SAGETIS BIOTECH, SL, Barcelona (ES)

(72) Inventors: Salvador Borros Gomez, Barcelona (ES); Francesc Xavier Rivero Monso, Barcelona (ES); Anna Cascante Cirera, Barcelona (ES)

(21) Appl. No.: 14/442,908

(22) PCT Filed: Nov. 14, 2013
(86) PCT No.: PCT/IB2013/060137

§ 371 Date: May 14, 2015

(30) Foreign Application Priority Data
Nov. 14, 2012 (GB) 1220474.9

(51) Int. Cl. C07K 14/00

(52) U.S. Cl. CPC C07K 14/001

(57) ABSTRACT

The present invention provides polypeptides that cross the blood brain barrier (BBB). These polypeptides are therefore BBB transport agents. The polypeptides are typically able to cross the BBB at a level effective to be therapeutically or diagnostically useful or physiologically significant, either alone or when coupled to a therapeutic or diagnostic agent.

### FIELD OF THE INVENTION

This invention relates to the delivery of agents to the brain. More particularly, the invention relates to polypeptides that can cross the Blood Brain Barrier (BBB) and their use in transporting an agent across the BBB, typically in the treatment and/or diagnosis of a neurological disease.

### BACKGROUND TO THE INVENTION

The BBB acts very effectively to protect the brain by restricting the entry of microscopic objects, such as bacteria and large or hydrophilic molecules, into the cerebral tissues. This is a major issue to take into account when developing drugs that target the central nervous system. To reach target cells in the cerebral tissues, a peripherally-administered drug must be capable of crossing the BBB by itself or by using a carrier as the transporting system. The BBB hinders delivery of many potentially important diagnostic and therapeutic agents to the brain, and so presents a major obstacle for the diagnosis and/or treatment of brain disorders.

A number of different strategies have been employed to help overcome the limitations imposed by the BBB, and these broadly fall into three categories: 1) invasive procedures (e.g. direct intraventricular administration of drugs by surgery); 2) pharmacological approaches (e.g. by increasing the lipid solubility of polypeptides); and 3) carrier-based approaches (i.e. by exploiting or modifying known carrier mechanisms to transport drugs across the BBB, providing highly specific and efficacious drug delivery).

In 2007, Demeule and co-workers designed a series of 19-mer polypeptides which were able to cross the BBB [1]. The best of these polypeptides, called “angiopep-2”, was shown to exhibit transcytosis in brain cells (transportation of molecules across the interior of the cells) through a binding mechanism involving the LDL receptor-related protein (LRP1) [2], which is a member of the low density lipoprotein receptor (“LDLR”) family.

LDLR is primarily responsible for the uptake of cholesterol-carrying particles into cells [3] through endocytosis. Its primary ligand is the low density lipoprotein (LDL) which is one of the 5 major groups of lipoproteins that enable transport of different fat molecules, including cholesterol. Approximately 65-70% of plasma cholesterol in humans circulates in the form of LDL. Each LDL particle contains a single apolipoprotein B100 molecule (apoB-100) which is responsible for the circulation of the fatty acids, keeping them soluble in the aqueous environment of the blood stream. LDLR also binds tightly to beta-migrating forms of very low-density lipoprotein (b-VLDL), which contain multiple copies of apolipoprotein E (apoE).

LDLR is a modular transmembrane protein of ˜840 amino acids which is representative of an entire class of receptors commonly denoted the LDLR family. Each LDLR family member contains one or several of the following domains arranged in a similar pattern: LDL receptor type-A (also denoted “LA”, “CR” or “ligand binding repeat”); epidermal growth factor-like (EGF-like) and YWTD (or β-propeller) modules (FIG. 1, [4]). The ectodomain of LDLR (FIG. 2, [4]) is functionally divided into 2 regions: a ligand-binding area consisting of 7 contiguous LDL-A modules (LA1-LA7)at the N-terminal end, and a subsequent region homologous to the EGF precursor. Modules LA3 to LA7 are essential for binding LDL [5]. The EGF region, which includes 2 EGF-like modules, a (3-propeller domain and a third EGF-like module, is responsible for both the release of the LDL particles at lower endosomal pH and the recycling of the receptor back to the cell surface.

To date, despite this knowledge, there is a limited choice when looking to transport agents across the BBB. There remains a need in the art for further and improved agents and methods to deliver therapeutic and diagnostic agents across the BBB.

### SUMMARY OF THE INVENTION

The present invention provides polypeptides that cross the BBB. These polypeptides are therefore BBB transport agents. The polypeptides are typically able to cross the BBB at a level effective to be therapeutically or diagnostically useful or physiologically significant, either alone or when coupled to a therapeutic or diagnostic agent.

Although the invention is not bound by theory, the polypeptides of the invention have been designed to bind the LA domain of LDLR, which internalises the polypeptides of the invention into the brain by endocytosis. Being able to cross the BBB, polypeptides of the invention are particularly useful as carriers for transporting other agents across the BBB and for delivering agents, typically drugs or diagnostic agents, to the brain.

The invention provides a polypeptide for crossing the blood brain barrier (BBB), wherein the polypeptide is, comprises, consists essentially of, or consists of:
(a) a regulon polypeptide less than 59 amino acids in length, comprising 7 or more consecutive amino acids of SEQ ID NO: 2, and comprising K48 and R49 (numbered relative to SEQ ID NO: 1), optionally comprising: (i) T43, V44, 145, H46, G47 and/or (ii) E50, V51, T52, L53 and H54 (numbered relative to SEQ ID NO: 1); optionally P43 and L55 (numbered relative to SEQ ID NO: 1); and optionally (i) P37, M38, A39, R40, E41 and/or (ii) H56, P57, D58, and H59, (numbered relative to SEQ ID NO: 1);
(b) a RAP polypeptide less than 100 amino acids in length comprising at least 20 consecutive amino acids from SEQ ID NO: 4;

(c) a flexible polypeptide less than 100 amino acids in length, comprising a flexible loop and wherein the polypeptide comprises the sequence: X₁ X₂ E X₃ X₄ X₅ X₆ R G K R X₇ X₈ X₉ K D E X₁₀ X₁₁ or RGKR X₈ X₉ K D E, wherein X₁=A, F, S or T; X₂=G, K, R or S; X₃=S or T;X₄=N or S; X₅=A, I or T; X₆=I, T or V; X₇=D, E or G; X₈=S, T or Y; X₉=F, T or Y; X₁₀=G or N; X₁₁=K or R; or
(d) a rigid polypeptide less than 100 amino acids in length, comprising an alpha helix and comprises the consensus sequence: (K/R) A (A/E/Q) K A (A/E/Q) A (K/R), optionally G D (A/E)_{α}(K/R) A (A/E/Q) K A (A/E/Q) A (K/R) A X_{β} G Y, wherein optionally ₆₀, is 1-10, and β is 1-25.

Preferably, a regulon polypeptide comprises or consists of SEQ ID NO: 2 or SEQ ID NO: 3.

Preferably, a RAP polypeptide comprises or consists of SEQ ID NO: 4, SEQ ID NO: 5 or SEQ ID NO: 6.

Preferably, a flexible polypeptide comprises or consists of SEQ ID NO: 7, SEQ ID NO: 8, SEQ ID NO: 9, SEQ ID NO: 10, or SEQ ID NO: 11.

Preferably, a rigid polypeptide comprises or consists of SEQ ID NO: 12, SEQ ID NO: 13, SEQ ID NO: 14, SEQ ID NO: 15, or SEQ ID NO: 16.

Polypeptides comprising or consisting of a sequence having at least 85%, 90% or 95% sequence identity to each of SEQ ID Nos. 2 to 16 are also provided.

In some embodiments, the polypeptide is produced recombinantly. In other embodiments, the polypeptide is chemically synthesised.

The invention also provides a conjugate for transporting an agent across the blood brain barrier (BBB), comprising: (a) a polypeptide as described above; and (b) an agent; wherein the conjugate is able to cross the BBB.

In some embodiments, the agent is a diagnostic agent or a therapeutic agent. In some embodiments, the polypeptide is conjugated to the agent via a linker. In some embodiments, the polypeptide is conjugated directly to the agent. In some embodiments, the conjugate comprises a nanoparticle. In some embodiments, the agent is releasable from the polypeptide after transport across the BBB.

The invention also provides a pharmaceutical composition comprising a polypeptide or conjugate according to any one of the preceding embodiments, and a pharmaceutically acceptable carrier, diluent or excipient.

The invention also provides a polypeptide, conjugate, or pharmaceutical composition as defined above, for use in therapy. In one embodiment, the therapeutic use is treating a neurological disease, optionally a brain tumor, brain metastasis, schizophrenia, epilepsy, Alzheimer's disease, Parkinson's disease, Huntington's disease, stroke, and/or disease associated with malfunction of the BBB.

The invention also provides a polypeptide, conjugate, or pharmaceutical composition as described above for use in a method of diagnosis. In one embodiment, the diagnostic use is diagnosis of a neurological disease.

The invention also provides an isolated polynucleotide encoding a polypeptide as described above.

### BRIEF DESCRIPTION OF DRAWINGS

The invention is described with reference to the following drawings.

FIG. 1: LDLR family members.

FIG. 2: Ectodomain of LDLR composed of 7 ligand-binding repeats (LA/CR), 3 EGF-like modules and a β-propeller unit.

FIG. 3: LA domain folding in LDLR (PDB:2 FCW)

FIG. 4: Comparison of LA-RAP(D3) interface (A-D) with other LA module interface.

FIG. 5: Hierarchical clustering of angiopeps based on a set of 113 physicochemical properties defined for each constituting AA.

FIG. 6: General folding of angiopep-2 obtained through homology modelling.

FIG. 7: Ab-initio prediction of AP2 structure.

FIG. 8: Possible interaction between AP2 (as single domain binder) and LA module.

FIG. 9: Possible interaction between AP2 (as double domain binder) and LA module.

FIG. 10: General folding of regulon according to its homology model (PDB code:3N40). The circle highlights the structured part of folding.

FIG. 11: Possible interaction between regulon (as single domain binder) and LA module.

FIG. 12: Possible interaction between regulon (as double domain binder) and LA module. Only the second binding region is shown.

FIG. 13: Predicted structure of regulon_construct1 as observed in the full regulon model.

FIG. 14: Predicted structure of regulon_construct1 calculated with an ab-initio method.

FIG. 15: Predicted structure of regulon_construct4 on the homology-based regulon model.

FIG. 16: Predicted structure of regulon_construct4 on a model calculated with an ab-initio method.

FIG. 17: Interaction between RAP D3 domain and LDLR.

FIG. 18: Model of RH_construct1 based on the crystal structure of RAP-LDLR complex (2FCW).

FIG. 19: Predicted structure of RH_construct1 calculated with an ab-initio method.

FIG. 20: Model of RH_construct2 based on the crystal structure of RAP-LDLR complex (2FCW).

FIG. 21: Model of RH_construct3 based on the crystal structure of RAP-LDLR complex (2FCW).

FIG. 22: Schematic representation of an in vitro model of the BBB, involving co-culture of bovine brain endothelial cells and astrocytes.

FIG. 23: Bar chart showing that un-decorated nanoparticles do not cross the BBB, whereas nanoparticles decorated with regulon (SEQ ID No. 1) do cross the BBB (NP=nanoparticle).

FIG. 24: Bar chart showing the percentage of crossing according to the fluorescently-labelled peptide in vitro BBB model crossing test.

### DETAILED DESCRIPTION OF THE INVENTION

The present invention is based on the surprising identification of polypeptides that can cross the BBB. In an attempt to identify new polypeptides that can cross the BBB, the inventors selected low density lipoprotein receptor (LDLR) as a target and compiled a database of all polypeptides known to interact with the ligand binding (“LA”) domain of LDLR. The inventors assessed the structure and activity of all of these polypeptides. Many of the polypeptide-LDLR interactions were identified using different experimental conditions, yet the inventors were able to identify structural and pharmacophoric features that are important for a polypeptide to be able to interact with the LA domain of LDLR, and so cross the BBB.

To develop and refine the structural and pharmacophoric model, the inventors analysed several peptides already known to cross the BBB, namely the angiopeps (Demeule et al, supra) and regulon. The angiopeps are known to cross the BBB by interacting with the LDLR, but no structural information was available for these polypeptides and so the inventors modelled the angiopeps to allow structural comparisons to be made. Regulon is also known to cross the BBB and its structure is known, but the mechanism by which regulon crosses the BBB was unknown. Having identified key structural and pharmacophoric features required for passing the BBB, the inventors developed the polypeptides of the invention, which contain these key features and so are also able to cross the BBB.

Polypeptides of the invention share common features. They are all able to cross the BBB, they were all identified by the same method and they are all designed to bind to the LA domain of LDLR.

Polypeptides of the invention can be sub-divided into four groups: “regulon polypeptides”, “RAP polypeptides”, “flexible polypeptides” and “rigid polypeptides”. Polypeptides of the invention typically comprise or consist of the sequences described below.

Regulon polypeptides
(SEQ ID NO: 1): Regulon
HKKWQFNSPFVPRADEPARKGKVHIPFPLDNITCRVPMAREPTVIHGKRE
VTLHLHPDH

Regulon is able to cross the BBB. Its structure and mechanism of action were, until now, unknown.

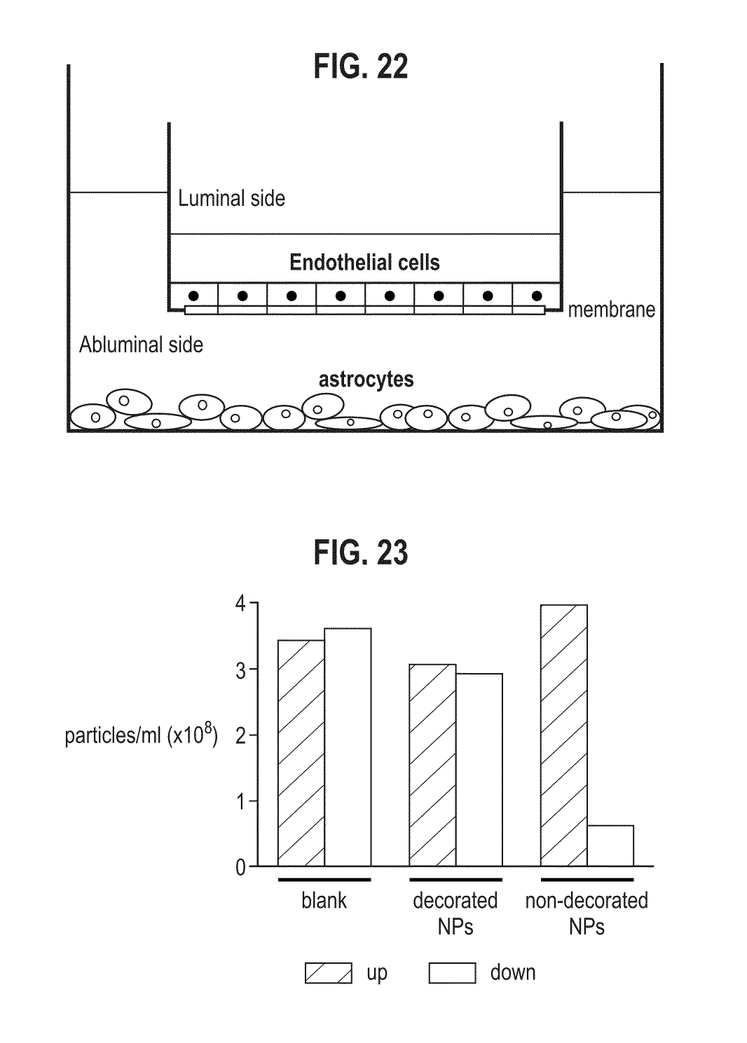

By homology modelling the Regulon sequence with the P62 envelope glycoprotein (discussed in the Examples below), the inventors found that regulon comprises a rigid β-hairpin structure and a long unstructured flexible chain. The inventors identified that the β-hairpin interacts with LDLR and found that this interaction requires 2 key residues, K48 and R49 (numbered relative to SEQ ID NO: 1), at the U-turn of the β-hairpin. Accordingly, regulon polypeptides of the invention comprise K48 and R49 (numbered relative to SEQ ID NO: 1). Regulon polypeptides of the invention are able to cross the BBB. This is shown, for example, by FIG. 23, which demonstrates that nanoparticles decorated with regulon (SEQ ID No. 1) are able to cross the BBB using a BBEC/rat astrocyte co-culture in vitro model as depicted in FIG. 22 and as described by Cecchelli et al, Adv Drug Deliv Rev. 1999 Apr 5;36(2-3):165-178. (reference 6). FIG. 23 also confirms that undecorated nanoparticles do not cross the BBB.

The full length regulon polypeptide is 59 AA long (SEQ ID NO: 1). Regulon polypeptides of the invention are fragments of full length regulon, and so are easier and cheaper to produce. Also, using shorter polypeptides to achieve the same or better passage across the BBB allows administration of a smaller weight of polypeptide to a patient. Accordingly, regulon polypeptides of the invention are less than 59 amino acids in length, and may therefore comprise 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, or 58 consecutive amino acids of SEQ ID NO: 1.

In addition to K48, and R49, regulon polypeptides of the invention preferably comprise (a) T43, V44, 145, H46 and G47 and/or (b) E50, V51, T52, L53 and H54 (numbered relative to SEQ ID NO: 1), because these amino acid residues are involved in forming the J3-hairpin. In addition to K48 and R49, regulon polypeptides of the invention more preferably comprise (a) P42, T43, V44, 145, H46 and G47 and/or (b) E50, V51, T52, L53, H54 and L55 (numbered relative to SEQ ID NO: 1), because these amino acid residues help form a larger β-hairpin motif that interacts with the LA domain of LDLR. Although not bound by theory, the inventors believe that this larger β-hairpin further improves interaction with the LDLR.

Regulon_construct1
(SEQ ID NO: 2): Regulon_construct1
PTVIHGKREVTLHL

A preferred regulon polypeptide of the invention is “regulon_construct1” (SEQ ID NO: 2). Regulon_construct1 is a 14 amino acid residue fragment of SEQ ID NO: 1, that forms a β-hairpin.

The invention also provides fragments of regulon_construct 1, that comprise 7 or more consecutive amino acids of SEQ ID NO: 2, e.g. 7, 8, 9, 10, 11, 12, or 13 amino acid residues, wherein the fragment comprises K48 and R49 (numbered relative to SEQ ID NO: 1), and retains the ability to cross the BBB.

Other preferred fragments lack one or more amino acids, e.g. 1, 2, 3, 4, 5 or 6 amino acids, from the C-terminus and/or one or more amino acids, e.g. 1, 2, 3, 4, 5 or 6 amino acids, from the N-terminus of SEQ ID NO: 2 while retaining the ability to cross the BBB; and wherein the fragment comprises 7 or more consecutive amino acids of SEQ ID NO: 2 and comprises K48 and R49 (numbered relative to SEQ ID NO: 1). Amino acid fragments of regulon_construct1 may thus comprise an amino acid sequence of 7, 8, 9, 10, 11, 12, or 13 consecutive amino acid residues of SEQ ID NO: 2.

Regulon polypeptides of the invention also include variants of SEQ ID NO: 2. Variants of regulon_construct1 typically consist of an amino acid sequence having 85% or more identity, e.g. 85%, 86%, 87%, 88%, 89%, 90%, 91%, 92%, 93%, 94%, 95%, 96%, 97%, 98%, 99% identity to SEQ ID NO: 2.

Regulon_construct4
(SEQ ID NO: 3): Regulon_construct4
PMAREPTVIHGKREVTLHLHPDH

Another preferred regulon polypeptide of the invention is “regulon_construct4” (SEQ ID NO: 3). Regulon_construct4 is a 23 amino acid residue fragment of SEQ ID NO: 1, which comprises SEQ ID NO: 2. SEQ ID NO: 4 is identified as particularly suitable because the additional residues (relative to SEQ ID NO: 2) form parallel strands that form a beta-sheet that can interact with the LDLR.

The invention also provides fragments of regulon_construct4, that comprise 7 or more consecutive amino acids of SEQ ID NO: 3, typically 7, 8, 9, 10, 11, 12, 13, 14, 15, 16, 17, 18, 19, 20, 21 or 22 amino acid residues, wherein the fragment comprises K48 and R49 (numbered relative to SEQ ID NO: 1), and retains the ability to cross the BBB.

Preferably, regulon_construct4 polypeptides of the invention comprise, in addition to K48 and R49, (a) P37, M38, A39, R40, E41, P42, T43, V44, 145, H46, and G47 and/or (b) E50, V51, T52, L53, H54, L55, H56, P57, D58, and H59 (numbered relative to SEQ ID NO: 1).

Other preferred fragments lack one or more amino acids, e.g. 1, 2, 3, 4, 5, 6, 7, 8, 9, 10, or 11 amino acids, from the C-terminus and/or one or more amino acids, e.g. 1, 2, 3, 4, 5, 6, 7, 8, 9, or 10 amino acids, from the N-terminus of SEQ ID NO: 3 while retaining the ability to cross the BBB, and wherein the fragment comprises 7 or more consecutive amino acids of SEQ ID NO: 3 and comprises K48 and R49 (numbered relative to SEQ ID NO: 1). Fragments of regulon_construct4 may thus comprise an amino acid sequence of 7, 8, 9, 10, 11, 12, 13, 14, 15, 16, 17, 18, 19, 20, 21, 22, or 23 consecutive amino acid residues of SEQ ID NO: 3.

Regulon polypeptides of the invention also include variants of SEQ ID NO: 3. Variants of regulon_construct4 typically consist of an amino acid sequence having 85% or more identity, e.g. 85%, 86%, 87%, 88%, 89%, 90%, 91%, 92%, 93%, 94%, 95%, 96%, 97%, 98%, 99% identity to SEQ ID NO: 3.

RAP polypeptides
(SEQ ID NO: 48): Receptor Associated Protein
MAPRRVRSFLRGLPALLLLLLFLGPWPAASHGGKYSREKNQPKPSPKRES
GEEFRMEKLNQLWEKAQRLHLPPVRLAELHADLKIQERDELAWKKLKLDG
LDEDGEKEARLIRNLNVILAKYGLDGKKDARQVTSNSLSGTQEDGLDDPR
LEKLWHKAKTSGKFSGEELDKLWREFLHHKEKVHEYNVLLETLSRTEEIH
ENVISPSDLSDIKGSVLHSRHTELKEKLRSINQGLDRLRRVSHQGYSTEA
EFEEPRVIDLWDLAQSANLTDKELEAFREELKHFEAKIEKHNHYQKQLEI
AHEKLRHAESVGDGERVSRSREKHALLEGRTKELGYTVKKHLQDLSGRIS
RARHNEL

Receptor Associated Protein (referred to herein as RAP, SEQ ID NO: 48) is known to be able to bind to the LDLR via its D3 domain and some structural studies have already been performed on the RAP protein. The interaction between RAP and LDLR is known to occur through 2 RAP alpha-helices which interact with 2 LDL receptor type-A (LA) modules. A single alpha-helix contains the essential residues for the interaction, including K256, while the other helix is believed to stabilize the complex. The inventors developed 3 new RAP polypeptides, which are termed “RH_construct1” (SEQ ID NO: 4), “RH_construct2” (SEQ ID NO: 5) and “RH_construct3” (SEQ ID NO: 6). The inventors identified RH_construct1 as a core fragment for crossing the BBB. Accordingly, “RH_construct2” and “RH_construct3” comprise the sequence of “RH_construct1”. “RH_construct3” also comprises “RH construct2”.

RAP polypeptides of the invention are fragments of full length RAP, and so are easier and cheaper to produce. Also, using shorter polypeptides to achieve the same or better passage across the BBB allows administration of a smaller weight of polypeptide to a patient. RAP polypeptides of the invention are less than 100 amino acids in length, typically 99, 98, 97, 96, 95, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, or 15 amino acids in length.

RAP polypeptides of the invention form an alpha-helix, and are able to cross the BBB.

RH_construct1
(SEQ ID NO: 4): RH_construct2
ELKHFEAKIEKHNHYQKQLE

“RH_construct1 (SEQ ID NO: 4) is a 20 amino acid residue fragment of RAP, which was identified as the minimal unit of RAP-D3 for interacting with LDLR.

RAP polypeptides of the invention also include variants of SEQ ID NO: 4. Variants of SEQ ID NO: 4 preferably consist of an amino acid sequence having 85% or more identity e.g. 85%, 86%, 87%, 88%, 89%, 90%, 91%, 92%, 93%, 94%, 95%, 96%, 97%, 98%, 99% identity to SEQ ID NO: 4.

RH_construct2
(SEQ ID NO: 5): RH_construct2
DKELEAFREELKHFEAKIEKHNHYQKQLEIAHEKLRHAESV

Another preferred RAP polypeptide of the invention is RH_construct2. RH_construct2 (SEQ ID NO: 5) is a 41 amino acid residue fragment of RAP which comprises SEQ ID NO: 4, and further favours α-helix formation.

RH_construct2 polypeptides of the invention comprise 20 or more consecutive amino acids of SEQ ID NO: 4, typically 20, 21, 22, 23, 24, 25, 26, 27, 28, 29, 30, 31, 32, 33, 34, 35, 36, 37, 38, 39 or 40 amino acid residues.

Other preferred fragments lack one or more amino acids, e.g. 1, 2, 3, 4, 5, 6, 7, 8, 9, 10, 11, 12, 13, or 14 amino acids, from the C-terminus and/or one or more amino acids, e.g. 1, 2, 3, 4, 5, 6, 7, 8, 9, 10, 11, 12, 13, 14, 15, 16 or 17 amino acids, from the N-terminus of SEQ ID NO: 5 while retaining the ability to cross the BBB, and wherein the fragment comprises 20 or more consecutive amino acids of SEQ ID NO: 4.

RAP polypeptides of the invention also include variants of SEQ ID NO: 5. Variants of RH_construct2 typically consist of an amino acid sequence having 85% or more identity e.g. 85%, 86%, 87%, 88%, 89%, 90%, 91%, 92%, 93%, 94%, 95%, 96%, 97%, 98%, 99% identity to SEQ ID NO: 5.

RH_construct3
(SEQ ID NO: 6): RH_construct3
DKELEAFREELKHFEAKIEKHNHYQKQLEIAHEKLRHAESVGDGERVSRS
REKHALLEGRTKELGYTVKKHLQDLSGRISRARH

Another preferred RAP polypeptide of the invention is RH_construct3. RH_construct3 (SEQ ID NO: 6) is an 84 amino acid fragment of RAP which comprises SEQ ID NO: 5 (and therefore also comprises SEQ ID NO: 4). RH_construct3 includes most of the RAP D3 domain, which comprises 2 α-helices. In addition to the α-helix of the RH_construct2 polypeptide, including the residues essential for the interaction with LDLR, RH_construct3 comprises a second α-helix containing Arg296 that interacts with LDLR and stabilizes the complex.

RH_construct3 polypeptides of the invention comprise 20 or more consecutive amino acids of SEQ ID NO: 4, typically 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 or 81 amino acid residues.

Other preferred fragments lack one or more amino acids, e.g. 1, 2, 3, 4, 5, 6, 7, 8, 9, 10, 11, 12, 13, or 14 amino acid residues, from the C-terminus and/or one or more amino acids, e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acid residues, from the N-terminus of SEQ ID NO: 6 while retaining the ability to cross the BBB, and wherein the fragment comprises 20 or more consecutive amino acids of SEQ ID NO: 4.

RAP polypeptides of the invention also include variants of SEQ ID NO: 6. Variants of RH_construct3 typically consist of an amino acid sequence having 85% or more identity, e.g. 85%, 86%, 87%, 88%, 89%, 90%, 91%, 92%, 93%, 94%, 95%, 96%, 97%, 98%, 99% identity to SEQ ID NO: 6.

Flexible Polypeptides

“Flexible polypeptides” comprise a flexible loop. The inventors identified features in the “AP2 double binder model” that interact with the LA domain of LDLR (see the Examples below). The flexible polypeptides of the invention contain these features and interact with the LA module of LDLR.

The “RGKRX₇X₈X₉KDE” motif was identified by the inventors as important for interacting with the LA domain of LDLR, and so flexible polypeptides of the invention comprise the amino acid sequence:

(SEQ ID NO: 23)
X₁ X₂ E X₃ X₄ X₅ X₆ R G K R X₇ X₈ X₉ K D
E X₁₀ X₁₁;
or
(SEQ ID NO: 24)
R G K R X₇ X₈ X₉ K D E

wherein:
X₁ preferably forms a hydrophobic intramolecular interaction with the amino acid at position 14, to promote the doubled Kunitz-type folding.
X₂ preferably has a high propensity to form a flexible-loop.
E promotes H-bonding intermolecular interaction with R103 of LA module.
X₃ is preferably a polar residue with high propensity to form a flexible-loop.
X₄ is preferably a polar residue with high propensity to form a flexible-loop.
X₅ preferably promotes a hydrophobic intermolecular interaction with V106 of the LA module.
X₆ preferably promotes hydrophobic intermolecular interaction with T126 of the LA module.
R G K R are considered essential for the interaction with LA module.
X₇ preferably has high propensity to form a flexible-loop.
X₈ is preferably a polar residue with high propensity to form a flexible-loop.
X₉ preferably promotes a hydrophobic intramolecular interaction with amino acid X₁ to promote the doubled Kunitz-type folding.
K is a AP2 residue considered essential for the interaction with LA module.
D promotes an H-bonding intermolecular interaction with Q104 of LA module.
E is a AP2 residue considered essential to interact intramolecularly with N-end and promote the doubled Kunitz-type folding.
X₁₀ is preferably a polar residue with high propensity to form a flexible-loop.
X₁₁ is preferably a polar residue that forms H-bonding intermolecular interaction with D110 of LA module.

Preferably, X₁=A, F, S or T
X₂=G, K, R or S
X₃=S or T
X₄=N or S
X₅=A, I or T
X₆=I, T or V
X₇=D, E or G
X₈=S, T or Y
X₉=F, T or Y
X₁₀=G or N
X₁₁=K or R

Flexible polypeptides of the invention may comprise SEQ ID NO: 24 and one or more of X₁ X₂ E X₃ X₄ X₅ X₆ X₁₀ and/or X₁₁ (of SEQ ID NO: 23). Flexible polypeptides of the invention may comprise:

(SEQ ID NO: 25)
X₂ E X₃ X₄ X₅ X₆ R G K R X₇ X₈ X₉ K D E X₁₀ X₁₁
(SEQ ID NO: 26)
E X₃ X₄ X₅ X₆ R G K R X₇ X₈ X₉ K D E X₁₀ X₁₁
(SEQ ID NO: 27)
X₃ X₄ X₅ X₆ R G K R X₇ X₈ X₉ K D E X₁₀ X₁₁
(SEQ ID NO: 28)
X₄ X₅ X₆ R G K R X₇ X₈ X₉ K D E X₁₀ X₁₁
(SEQ ID NO: 29)
X₅ X₆ R G K R X₇ X₈ X₉ K D E X₁₀ X₁₁
(SEQ ID NO: 30)
X₆ R G K R X₇ X₈ X₉ K D E X₁₀ X₁₁
(SEQ ID NO: 31)
R G K R X₇ X₈ X₉ K D E X₁₀ X₁₁
(SEQ ID NO: 32)
X₁ X₂ E X₃ X₄ X₅ X₆ R G K R X₇ X₈ X₉ K D E X₁₀
(SEQ ID NO: 33)
X₂ E X₃ X₄ X₅ X₆ R G K R X₇ X₈ X₉ K D E X₁₀
(SEQ ID NO: 34)
E X₃ X₄ X₅ X₆ R G K R X₇ X₈ X₉ K D E X₁₀
(SEQ ID NO: 35)
X₃ X₄ X₅ X₆ R G K R X₇ X₈ X₉ K D E X₁₀
(SEQ ID NO: 36)
X₄ X₅ X₆ R G K R X₇ X₈ X₉ K D E X₁₀
(SEQ ID NO: 37)
X₅ X₆ R G K R X₇ X₈ X₉ K D E X₁₀
(SEQ ID NO: 38)
X₆ R G K R X₇ X₈ X₉ K D E X₁₀
(SEQ ID NO: 39)
R G K R X₇ X₈ X₉ K D E X₁₀
(SEQ ID NO: 40)
X₁ X₂ E X₃ X₄ X₅ X₆ R G K R X₇ X₈ X₉ K D E
(SEQ ID NO: 41)
X₂ E X₃ X₄ X₅ X₆ R G K R X₇ X₈ X₉ K D E
(SEQ ID NO: 42)
E X₃ X₄ X₅ X₆ R G K R X₇ X₈ X₉ K D E
(SEQ ID NO: 43)
X₃ X₄ X₅ X₆ R G K R X₇ X₈ X₉ K D E
(SEQ ID NO: 44)
X₄ X₅ X₆ R G K R X₇ X₈ X₉ K D E
(SEQ ID NO: 45)
X₅ X₆ R G K R X₇ X₈ X₉ K D E
(SEQ ID NO: 46)
X₆ R G K R X₇ X₈ X₉ K D E
(SEQ ID NO: 47)
R G K R X₇ X₈ X₉ K D E.

Shorter polypeptides are cheaper and easier to produce. Also, using shorter polypeptides to achieve the same or better passage across the BBB allows administration of a smaller weight of polypeptide to a patient.

Flexible polypeptides of the invention are less than 100 amino acids in length, typically 99, 98, 97, 96, 95, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11 or 10 amino acids in length. Preferably, flexible polypeptides of the invention are 15-24, i.e. 15, 16, 17, 18, 19, 20, 21, 22, 23 or 24 amino acids in length, more preferably 18-21 amino acids in length, i.e. 18, 19, 20 or 21 amino acids in length.

Most preferably, flexible polypeptides of the invention are 19 amino acids in length.

Flexible polypeptides of the invention also include variants of the flexible polypeptides listed above. Flexible polypeptides of the invention typically consist of an amino acid sequence having 85% or more identity, e.g. 85%, 86%, 87%, 88%, 89%, 90%, 91%, 92%, 93%, 94%, 95%, 96%, 97%, 98%, 99% identity, to one or more of the flexible polypeptides listed above.

Preferably, flexible polypeptides of the invention comprise, or consist of SEQ ID NO: 7, 8, 9, 10 or 11 and fragments or variants thereof.

Preferred rigid polypeptides of the invention are SEQ ID NO: 7 (“flex_{—}1”), SEQ ID NO: 8 (“flex_{—}2”), SEQ ID NO: 9 (“flex_{—}3”), SEQ ID NO: 10 (“flex_{—}4”) or SEQ ID NO: 11 (“flex_{—}5”), as well as fragments and/or variants thereof.

“flex_1”
(SEQ ID NO: 7): flex_1
T G E S N T V R G K R G S Y K D E N R

In some embodiments, flexible polypeptides of the invention (i) consist of SEQ ID NO: 7; (ii) comprise the amino acid sequence of SEQ ID NO: 7; (iii) have at least 85% identity to SEQ ID NO: 7, i.e. 85%, 86%, 87%, 88%, 89%, 90%, 91%, 92%, 93%, 94%, 95%, 96%, 97%, 98%, 99%; and/or (iv) contain 10 or more consecutive amino acids of SEQ ID NO: 7, i.e. 10, 11, 12, 13, 14, 15, 16, 17, 18 or 19 consecutive amino acids. Flex_{—}1 polypeptides of the invention are less than 100 amino acids in length.

“flex_2”
(SEQ ID NO: 8): flex_2
F R E S N T I R G K R E T T K D E N R

In some embodiments, flexible polypeptides of the invention (i) consist of SEQ ID NO: 8; (ii) comprise the amino acid sequence of SEQ ID NO: 8; (iii) typically have at least 85% identity to SEQ ID NO: 8, preferably at least 91% identity to SEQ ID NO: 8, i.e. 91%, 92%, 93%, 94%, 95%, 96%, 97%, 98%, 99%; and/or (iv) contain 9 or more consecutive amino acids of SEQ ID NO: 8, i.e. 9, 10, 11, 12, 13, 14, 15, 16, 17, 18 or 19 consecutive amino acids. Flex _{—}2 polypeptides of the invention are less than 100 amino acids in length.

“flex_3”
(SEQ ID NO: 9): flex_3
T K E T S A T R G K R E T T K D E G K

In some embodiments, flexible polypeptides of the invention (i) consist of SEQ ID NO: 9; (ii) comprise the amino acid sequence of SEQ ID NO: 9; (iii) typically have at least 85% identity to SEQ ID NO: 9, preferably at least 91% identity to SEQ ID NO: 9, i.e. 91%, 92%, 93%, 94%, 95%, 96%, 97%, 98%, 99%; and/or (iv) contain 9 or more consecutive amino acids of SEQ ID NO: 9, i.e. 9, 10, 11, 12, 13, 14, 15, 16, 17, 18 or 19 consecutive amino acids. Flex_{—}3 polypeptides of the invention are less than 100 amino acids in length.

“flex_4”
(SEQ ID NO: 10): flex_4
A R E T S I V R G K R D Y F K D E G K

In some embodiments, flexible polypeptides of the invention (i) consist of SEQ ID NO: 10; (ii) comprise the amino acid sequence of SEQ ID NO: 10; (iii) have at least 85% identity to SEQ ID NO: 7, i.e. 85%, 86%, 87%, 88%, 89%, 90%, 91%, 92%, 93%, 94%, 95%, 96%, 97%, 98%, 99%; and/or (iv) contain 9 or more consecutive amino acids of SEQ ID NO: 10, i.e. 9, 10, 11, 12, 13, 14, 15, 16, 17, 18 or 19 consecutive amino acids. Flex_{—}4 polypeptides of the invention are less than 100 amino acids in length.

“flex_5”
(SEQ ID NO: 11): flex_5
S S E S N I T R G K R E Y T K D E G R

In some embodiments, flexible polypeptides of the invention (i) consist of SEQ ID NO: 11; (ii) comprise the amino acid sequence of SEQ ID NO: 11; (iii) typically have at least 85% identity to SEQ ID NO: 11, preferably at least 90% identity to SEQ ID NO: 11, i.e. 90%, 91%, 92%, 93%, 94%, 95%, 96%, 97%, 98%, 99%; and/or (iv) contain 10 or more consecutive amino acids of SEQ ID NO: 11, i.e. 10, 11, 12, 13, 14, 15, 16, 17, 18 or 19 consecutive amino acids. Flex_{—}5 polypeptides of the invention are less than 100 amino acids in length.

Rigid Polypeptides

A further group of polypeptides identified by the inventors as being suitable for BBB transport is termed the “rigid polypeptides”. “Rigid polypeptides” of the invention are so named because their secondary structure comprises an α-helix and their design was based on the co-crystallized RAP-LA complex. Having identified the main interacting motif of RAP (discussed in the Examples below), the inventors then analysed this to provide further polypeptides that are able to cross the BBB. Rigid polypeptides of the invention represent an improvement over full length RAP because they are much shorter and thus cheaper and easier to produce. Also, using shorter polypeptides to achieve the same or better passage across the BBB allows administration of a smaller weight of polypeptide to a patient.

Rigid polypeptides of the invention comprise or consist of an amino acid sequence with the consensus sequence:

(K/R) A (A/E/Q) K A (A/E/Q) A (K/R)

Preferably, rigid polypeptides of the invention comprise or consist of an amino acid with the consensus sequence:

G D (A/E)₆₀ (K/R) A (A/E/Q) K A (A/E/Q) A (K/R) A X₆₂ G Y

wherein preferably, ₆₀ is 1-10, and ₆₂ is 1-25

Rigid polypeptides of the invention are less than 100 amino acids in length, typically 99, 98, 97, 96, 95, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11 or 10 amino acids in length. Rigid polypeptides of the invention are preferably 10-45 amino acids in length, i.e. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 amino acids.

Rigid polypeptides of the invention also include variants of the rigid polypeptides listed above. Rigid polypeptides of the invention preferably consist of an amino acid sequence: having 95% or more identity, e.g. at least 95%, 96%, 97%, 98%, 98.5% or 99% identity to one or more of the rigid polypeptides listed above.

Preferred rigid polypeptides of the invention are selected from the group consisting of SEQ ID NO: 12 (“rigid_{—}1”), SEQ ID NO: 13 (“rigid_{—}2”), SEQ ID NO: 14 (“rigid_{—}3”), SEQ ID NO: 15 (“rigid_{—}4”) or SEQ ID NO: 16 (“rigid_{—}5”), as well as fragments and/or variants thereof.

“rigid_1”
(SEQ ID NO: 12): rigid_1
GDAAAAKAAKAAAKAAADGY

The “rigid_{—}1” polypeptide (SEQ ID NO: 12) is identified as a single domain binder (i.e. that can interact with a single LA module of LDLR). In some embodiments, rigid polypeptides of the invention (i) consist of SEQ ID NO: 12; (ii) comprise the amino acid sequence of SEQ ID NO: 12; (iii) typically have at least 85% identity to SEQ ID NO: 12, preferably at least 95% identity to SEQ ID NO: 12, i.e. 95%, 96%, 97%, 98%, 99%; and/or (iv) contain 16 or more consecutive amino acids of SEQ ID NO: 12, i.e. 16, 17, 18, 19 or 20 consecutive amino acids. Rigid_{—}1 polypeptides of the invention are less than 100 amino acids in length (as discussed above).

“rigid_2”
(SEQ ID NO: 13): rigid_2
GDAAAARAAKAAARAAADGY

The “rigid_{—}2” polypeptide (SEQ ID NO: 13) is identified as single domain binder. In some embodiments, rigid polypeptides of the invention (i) consist of SEQ ID NO: 13; (ii) comprise the amino acid sequence of SEQ ID NO: 13; (iii) typically have at least 85% identity to SEQ ID NO: 13, preferably at least 95% identity to SEQ ID NO: 13, i.e. 95%, 96%, 97%, 98%, 99%; and/or (iv) contain 11 or more consecutive amino acids of SEQ ID NO: 13, i.e. 11, 12, 13, 14, 15, 16, 17, 18, 19 or 20 consecutive amino acids. Rigid_{—}2 polypeptides of the invention are less than 100 amino acids in length (as discussed above).

“rigid_3”
(SEQ ID NO: 14): rigid_3
GDAAAAKAAKAAAKAAAAAAKAAKAAAKAAADGY

The “rigid_{—}3” polypeptide (SEQ ID NO: 14) is identified as a double domain binder (i.e. that can interact simultaneously with two LA modules of LDLR). In some embodiments, rigid polypeptides of the invention (i) consist of SEQ ID NO: 14; (ii) comprise the amino acid sequence of SEQ ID NO: 14; (iii) have at least 85% identity to SEQ ID NO: 14, i.e. 85%, 86%, 87%, 88%, 89%, 90%, 91%, 92%, 93%, 94%, 95%, 96%, 97%, 98%, 99%; and/or (iv) contain 7 or more consecutive amino acids of SEQ ID NO: 14, i.e. 7, 8, 9, 10, 11, 12, 13, 14, 15, 16, 17, 18, 19, 20, 21, 22, 23, 24, 25, 26, 27, 28, 29, 30, 31, 32, 33 or 34 consecutive amino acids. Rigid_{—}3 polypeptides of the invention are less than 100 amino acids in length (as discussed above).

“rigid_4”
(SEQ ID NO: 15): rigid_4
GDAAEAKAEKAEAKAEAAEAKAEKAEAKAAAEGY

The “rigid_{—}4” polypeptide (SEQ ID NO: 15) is identified as a double domain binder. In some embodiments, rigid polypeptides of the invention (i) consist of SEQ ID NO: 15; (ii) comprise the amino acid sequence of SEQ ID NO: 15; (iii) have at least 85% identity to SEQ ID NO: 15, i.e. 85%, 86%, 87%, 88%, 89%, 90%, 91%, 92%, 93%, 94%, 95%, 96%, 97%, 98%, 99%; and/or (iv) contain 7 or more consecutive amino acids of SEQ ID NO: 15, i.e. 7, 8, 9, 10, 11, 12, 13, 14, 15, 16, 17, 18, 19, 20, 21, 22, 23, 24, 25, 26, 27, 28, 29, 30, 31, 32, 33 or 34 consecutive amino acids. Rigid_{—}4 polypeptides of the invention are less than 100 amino acids in length (as discussed above).

“rigid_5”
(SEQ ID NO: 16): rigid_5
GDAAEAKAQKAQAKANAAKAKAQKAQAKAAANGY

The “rigid_{—}5” polypeptide (SEQ ID NO: 16) is identified as a putative double domain binder. In some embodiments, rigid polypeptides of the invention (i) consist of SEQ ID NO: 16; (ii) comprise the amino acid sequence of SEQ ID NO: 16; (iii) have at least 85% identity to SEQ ID NO: 16, i.e. 85%, 86%, 87%, 88%, 89%, 90%, 91%, 92%, 93%, 94%, 95%, 96%, 97%, 98%, 99%; and/or (iv) contain 7 or more consecutive amino acids of SEQ ID NO: 16, i.e. 7, 8, 9, 10, 11, 12, 13, 14, 15, 16, 17, 18, 19, 20, 21, 22, 23, 24, 25, 26, 27, 28, 29, 30, 31, 32, 33 or 34 consecutive amino acids. Rigid_{—}5 polypeptides of the invention are less than 100 amino acids in length (as discussed above).

Assays for Crossing the Blood Brain Barrier

Polypeptides and conjugates of the invention can cross the BBB. The BBB may be in vivo, or may be an ex vivo or in vitro model of the BBB, as is known in the art. Assays to test whether a polypeptide or conjugate can cross the BBB are also well known in the art.

The skilled person will appreciate that suitable in vitro BBB models using cells of cerebral origin include: (i) isolated brain capillaries; (ii) primary or low passage brain capillary endothelial cell cultures; (iii) bovine brain endothelial cell culture (BBEC); and (iv) immortalized brain endothelial cells e.g. RBE4 cell line, RBEC1 cell line and TR-BBB13 cell line.

In vitro BBB models using cells of non-cerebral origin that are known in the art to be useful as a model of the BBB include MDCK (Madin-Darby canine kidney) cells and CaCo-2 cells.

In vivo models of the BBB include: (i) the carotid artery injection technique, wherein a test compound is injected into the common carotid artery of an animal with a radiolabaelled reference compound and the brain analysed seconds after injection; (ii) the in situ perfusion technique, which involves a longer experimental period with carotid artery perfusion of the brain followed by sampling of drug levels within the brain; (iii) the intravenous injection technique, in which a femoral vein or the tail vein is cannulated and the test compound injected with a plasma volume marker and arterial blood collected at various time points; and (iv) intracerebral microdialysis, which involves direct sampling of brain interstitial fluid by implanting into the brain a dialysis fibre perfused with a physiological solution, whereby compounds that enter the brain interstitial fluid will permeate into the physiological solution and can be assayed by an appropriate technique (HPLC, capillary electrophoresis).

It is therefore a straightforward task for the skilled person to determine whether a given compound is able to cross the blood brain barrier in a therapeutically useful or physiologically significant amount.

An example of a suitable in vitro BBB transport assay that can be used to confirm that a polypeptide or conjugate is able to cross the BBB, is a bovine brain endothelial cell culture (BBEC). In a BBEC model, cells are isolated from the grey matter of the bovine brain and are then typically grown on surfaces previously coated with rat tail collagen. In order to improve the model, BBEC can be co-cultured with primary astrocytes (typically from neonatal rats) or C6 rat glioma cells. FIG. 22 depicts the co-culture of astrocytes and bovine brain capillary endothelial cells. These co-culture assays provide a close resemblance to the BBB in vivo. Cecchelli et al (reference 6) describes a preferred BBEC assay that involves co-culture of bovine brain capillary endothelial cells and rat primary astrocytes. This assay has been shown to provide a legitimate in vitro model of the BBB, with a strong in vivo and in vitro correlation. In this model (in particular as described on pages 168-172 of reference 6), and as depicted in FIG. 22, bovine brain endothelial cells are cultured on a membrane and rat primary astrocytes are cultured on the bottom of the culture dish.

As shown in FIG. 22, the culture medium is shared by both cell populations, allowing humoral interchange without direct cell contact. FIG. 22 illustrates the structure of confluent bovine brain endothelial cells cultured on an insert coated with collagen (Millicell C M, 0.4-mm pore size, Millipore). Bovine brain capillary endothelial cells form a monolayer of small, tightly packed, non-overlapping and contact-inhibited cells. In these culture conditions, bovine brain capillary endothelial cells retain both endothelial (factor VIII-related antigen, angiotensin converting enzyme) and the BBB features. A kit (“CT Bovial@BBB Pack”) for performing this BBB transport assay is commercially available from Cellial (Lens, France; www.cellial.com).

Permeability and general mechanisms of transport can be elucidated by methods known in the art. In reference 6, for example, Ringer-HEPES (150 mM NaCl, 5.2 mM KCl, 2.2 mM CaCl , 0.2 mM MgCl, 6 mM NaHCO , 2.8 mM glucose, 5 mM HEPES) is added to the lower compartments of a six well plate (2.5 ml per well). One filter is then transferred into the first well of the six-well plate containing Ringer, and Ringer containing labeled or unlabelled drugs is placed in the upper compartment. At different times after addition of the drugs, the filter is transferred to another well of the six-well plate to minimize the possible passage from the lower to the upper compartment. Incubations are performed on a rocking platform at 37° C. Shaking minimizes the thickness of the aqueous boundary layer on the cell monolayer surface and influences the permeability of lipophilic solutes. An aliquot from each lower compartment and stock solution is taken and the amount of drugs in each sample is measured by HPLC for unlabelled drugs or in a liquid scintillation counter for labelled drugs. Permeability calculations may be performed as described by Siflinger-Birnboim et al., J Cell. Physiol. 132; 111-117 (reference 45).

The assay described above, or another suitable assay known in the art, can be used to confirm that polypeptides and conjugates of the invention are able to cross the blood-brain barrier.

Assay for Binding to the LDLR

Polypeptides and conjugates of the invention were designed to bind to the LDLR via its LA domain. Whether or not a polypeptide binds to the LDLR LA domain can be tested using standard techniques well known to the skilled person, for example an immunoassay such as an ELISA assay, a radioligand binding assay, a surface plasmon resonance assay such as the Biacore™ method, or a structural analysis (e.g. X-ray) with and without the test polypeptide. Typically, polypeptides of the invention bind to the LA domain of LDLR with at least a micromolar dissociation constant (K_{d}), e.g. at least 10⁻⁶M, preferably a nanomolar K_{d} e.g. at least 10⁻⁹M.

Anti-LDLR antibodies may be used in a competition assay to test whether a polypeptide or conjugate crosses the BBB by binding LDLR. Anti-LDLR antibodies compete for LDLR binding with polypeptides and conjugates that bind LDLR, and so reduce the amount of polypeptide or conjugate that binds the LDLR and passes the BBB. LDLR-mediated crossing of the BBB can therefore be determined by performing a BBB transport assay, as described above, in the presence or absence of anti-LDLR antibodies (that block binding to the receptor), and comparing the permeability of the BBB to the peptides and conjugates. A reduced passage across the BBB in the presence of an anti-LDLR antibody indicates that the polypeptide or conjugate crosses the BBB mediated by LDLR.

Glioma cells are described to overexpress the LRP1 (LDLR) receptor on their surface and this has been confirmed by contacting glioma cells (U87MG) with abcam LRP1 antibody (1:500 dilution) labelled with Alexa 488. Collocation was performed with DAPI and photos taken. This can be repeated with the confocal microscope and with bovine endothelial cells (BBB kit). Accordingly glioma cells express LRP1 and an in vitro BBB model comprising glioma cells, e.g. BBEC co-cultured with glioma cells, may be used in a competition assay (as described above) to confirm whether a polypeptide or conjugate crosses the BBB by binding LDLR.

Polypeptides of the Invention

Polypeptides comprise two or more amino acid residues linked by a peptide bond. The term “polypeptide” is used interchangeably with the term “peptide” and “protein”.

Polypeptides of the invention can take various forms, such as native, fusion, glycosylated, non-glycosylated, lipidated, non-lipidated, phosphorylated, non-phosphorylated, myristoylated, non-myristoylated, monomeric, multimeric, particulate, or denatured.

Polypeptides of the invention can be prepared by various means, including recombinant expression, purification from cell culture and chemical synthesis. Recombinantly-expressed polypeptides are preferred, particularly for hybrid and fusion polypeptides.

Polypeptides of the invention can be provided in purified or substantially purified form i.e. substantially free from other polypeptides, e.g. free from naturally-occurring polypeptides, particularly from other host cell polypeptides, and are generally at least about 50% pure (by weight), and usually at least about 90% pure i.e. less than about 50%, and more preferably less than about 10% (e.g. 5%) of a composition is made up of other expressed polypeptides. Thus the polypeptides in the compositions are separated from the whole organism with which the molecule is expressed.

Polypeptides of the invention are typically isolated or purified.

The term “polypeptide” refers to amino acid polymers of any length. The polymer may be linear or branched, it may comprise modified amino acids, and it may be interrupted by non-amino acids. The term also encompasses an amino acid polymer that has been modified naturally or by intervention; for example, disulfide bond formation, glycosylation, lipidation, acetylation, phosphorylation, or any other manipulation or modification, such as conjugation with a labelling component. Also included are, for example, polypeptides containing one or more analogs of an amino acid (including, for example, unnatural amino acids), as well as other modifications known in the art. Polypeptides can occur as single chains or associated chains.

Variants of polypeptides of the invention preferably include individual substitutions, deletions or additions to a polypeptide sequence that result in the substitution of an amino acid with a chemically similar amino acid. Tables describing functionally similar amino acids are well known in the art.

Conjugates In one aspect, the invention provides a polypeptide of the invention (i.e. a “regulon polypeptide”, “RAP polypeptide”, “flexible polypeptide” or “rigid polypeptide”; e.g. comprising any of SEQ ID Nos. 2 to 16) linked to an agent. This polypeptide linked to an agent is referred to as a conjugate, which is common terminology in the art. Conjugates of the invention are able to cross the BBB. Typically, the agent is an agent to be transported across the BBB, such as an agent for use in diagnosis or therapy.

The agent is typically a therapeutic agent or a diagnostic agent.

The agent may be a drug, a polypeptide, an enzyme, an antibiotic, an anti-cancer agent, a radioactive agent, an antibody, a cellular toxin, a detectable label or an anti-angiogenic compound.

In some embodiments the agent is a heterologous polypeptide, e.g. the invention provides a fusion protein containing a polypeptide of the invention and a different polypeptide. The skilled person will understand that when the polypeptide of the invention is a fragment of a longer polypeptide, the “heterologous protein” is not the reciprocal sequence of the longer polypeptide.

In some embodiments, the agent comprises or is part of a nanoparticle, which acts as a vehicle for the therapeutic and/or diagnostic agent. The polypeptide of the invention is typically linked to the surface of the nanoparticle. Preferably the nanoparticle is biodegradable. The therapeutic or diagnostic agent is typically dissolved in, entrapped in, encapsulated in or attached to a nanoparticle matrix. Biodegradable nanoparticles, particularly those coated with hydrophilic polymer such as poly(ethylene glycol) (PEG), are useful as drug delivery devices as they circulate for a prolonged period and may target a particular site for delivery (Mohanraj & Chen Trop. I Pharm. Res. 5, 561-573 (2006)). The advantages of using nanoparticles as a release vehicle are manifold. The particle size and surface characteristics of nanoparticles can be easily manipulated to achieve both passive and active targeting of a therapeutic or diagnostic agent after systemic passage. In particular, they control and sustain release of the therapeutic agent during the transportation and at the site of localization, altering organ distribution of the therapeutic agent and subsequent clearance of the therapeutic agent so as to achieve an increase in therapeutic efficacy and a reduction in side effects by minimising interaction with other organs. Controlled release and particle degradation characteristics can be readily modulated by the choice of matrix constituents. Loading of therapeutic or diagnostic agent is relatively high and therapeutic or diagnostic agents can be incorporated into the systems without any chemical reaction; this is an important factor for preserving the activity of the therapeutic or diagnostic agent.

In one embodiment, the nanoparticle is a nanoparticle as described in co-pending application PCT/IB2012/052320, wherein the nanoparticle comprises a block copolymer, and optionally one or more therapeutic or diagnostic agent(s),wherein:
(i) the block copolymer comprises blocks A and D;
(ii) block A consists of a first polymer comprising monomer units B and C, wherein B is an aliphatic dicarboxylic acid wherein the total number of carbon atoms is ≦30 and C is a dihydroxy or diamino monomer; and
(iii) block D consists of a second polymer comprising a hydrocarbon chain containing ester or ether bonds with hydroxyl number ≧10.

The therapeutic or diagnostic agent(s) can be present within the nanoparticles or on the surfaces of the nanoparticles. The interaction between the therapeutic or diagnostic agent(s) and the nanoparticle is typically non-covalent, for example, hydrogen bonding, electrostatic interactions or physical encapsulation. However, in an alternative embodiment, the therapeutic or diagnostic agent(s) and the nanoparticle are linked by a covalent bond or linker.

In some embodiments, the invention provides multimers of polypeptides of the invention conjugated to one or more agents.

The conjugates are typically able to cross the BBB at a level effective to be therapeutically or diagnostically useful, or at a physiologically significant level. The level of the therapeutic or diagnostic agent that is required will depend on the agent, the subject and the condition to be diagnosed or treated, and can readily be determined by the skilled person.

Typically, conjugates of the invention retain the ability of the polypeptide of the invention to cross the BBB, i.e., the conjugate has at least 70%, 75%, 80%, 85%, 90%, 95%, 100%, 105%, 110%, 115%, 120%, 125%, 130% of the ability to cross the BBB compared to the polypeptide of the invention prior to conjugation with agent. The ability to cross the BBB may be assessed using any suitable BBB transport assay, including the assays recited above.

Conjugates wherein the Agent is a Therapeutic Agent

In one embodiment, the agent to which the polypeptide of the invention is linked, is a therapeutic agent. The therapeutic agent is typically a drug.

The therapeutic agent may in certain embodiments be a small molecule drug, typically having a molecular weight of less than 1000 daltons. In other embodiments, the therapeutic agent may be a “biological” such as an antibody or antibody fragment, interleukin, interferon, or other protein.

In one embodiment, the invention provides a fusion protein containing a polypeptide of the invention and a heterologous (therapeutic) polypeptide.

Typical therapeutic agents include (i) chemotherapeutic agents, which may function as microtubulin inhibitors, mitosis inhibitors, topoisomerase inhibitors, or DNA intercalators; (ii) protein toxins, which may function enzymatically; (iii) radioisotopes; (iv) antibiotics; (v) analgesics; (vi) anti-psychotics; and (vii) anti-depressants. Therapeutic agents that are active within the CNS, more preferably in the brain, are preferred. These include (i) psychoactive drugs including anti-depressants, anti-psychotics, stimulants, anxiolytics and depressants, and (ii) antineoplastic drugs to treat a brain neoplasm, e.g. a brain tumour.

Conjugates wherein the Agent is a Diagnostic Agent

In one embodiment, the agent to which the polypeptide of the invention is linked, is a diagnostic agent. A diagnostic agent is an agent that can be used to determine the presence or extent of a disease, disorder, condition or pathology; preferably, a neurological disease is diagnosed. Typical diagnostic agents are x-ray contrast preparations, radioactive isotopes, labels and dyes.

The diagnostic agent typically comprises or consists of a dye, a chemi-luminescent dye, radioimaging agent, metal chelate complex, label e.g. fluorescent label, enzyme-substrate label, an antibody or an antibody fragment thereof.

The term “label” as used herein refers to a agent which can be attached to a polypeptide of the invention that functions to: (i) provide a detectable signal; (ii) interact with a second label to modify the detectable signal provided by the first or second label, e.g. FRET (fluorescence resonance energy transfer); (iii) stabilize interactions or increase affinity of binding, with antigen or ligand; (iv) affect mobility, e.g. electrophoretic mobility, or cell-permeability, by charge, hydrophobicity, shape, or other physical parameters, or (v) provide a capture moiety, to modulate ligand affinity, antibody/antigen binding, or ionic complexation.

In some embodiments, the diagnostic agent is a heterologous polypeptide e.g. the invention provides a fusion protein containing a polypeptide of the invention and a heterologous (diagnostic) polypeptide. The heterologous polypeptide may be labelled. The heterologous polypeptide may be an antibody, antibody fragment, receptor or other polypeptide able to selectively bind to a target, such as an altered protein characteristic of a disease pathology. For example, a labelled antibody or fragment that selectively binds to amyloid plaques could be used to diagnose Alzheimer's disease, while a labelled antibody or fragment that selectively binds to a tumour marker could be sued to diagnose the presence of a tumour.

Linking Polypeptides of the Invention to Agents

Polypeptides of the invention can be linked, i.e. conjugated, to agents by any suitable means known in the art. Typically, the linkage will be carried out by chemical conjugation or (when the agent is a polypeptide) by expressing a fusion protein comprising the polypeptide of the invention and the agent.

In some embodiments, the polypeptide of the invention is conjugated to the agent via a “linker”. Linkers have at least two reactive sites. One reactive site of the linker is bound to a residue of the polypeptide, and the other reactive site is bound to the agent. A linker is a bifunctional or multifunctional moiety which can be used to link one or more agents to a polypeptide of the invention to form a conjugate. Conjugates can be conveniently prepared using a linker having reactive functionality for binding to the agent and to the polypeptide of the invention. In some embodiments, for example wherein the conjugate comprises a polypeptide of the invention and a heterologous protein (i.e. the agent), the linker can be another polypeptide sequence positioned at the C-terminus of the polypeptide of the invention and at the N-terminus of the agent, or vice versa. In such cases, conjugates can be conveniently prepared by expressing the polypeptide of the invention, the heterologous protein (i.e. the agent) and the polypeptide linker as a hybrid or fusion protein.

In other embodiments, the polypeptide of the invention is conjugated directly to the agent (i.e. the polypeptide of the invention is not conjugated to the agent via a linker).

In some embodiments, the agent is a heterologous polypeptide. In such cases, the polypeptide of the invention is typically conjugated to the agent via a peptide bond. Peptide bonds can be prepared, for example, according to the liquid phase synthesis method (see E. Schroder and K. Luibke, “The polypeptides”, volume 1, pp 76-136, 1965, Academic Press) that is well known in the field of polypeptide chemistry.

Peptide bonds between polypeptides of the invention and heterologous polypeptides (and optionally a polypeptide linker) may be formed by expressing the conjugate as a hybrid or fusion polypeptide.

Conjugates comprising a polypeptide of the invention and an agent that is a heterologous polypeptide may be represented by the formula NH₂-F-A-{-X-L-}ₙ-B-G-COOH, wherein: X is an amino acid sequence of a polypeptide of the invention; L is an optional linker amino acid sequence; A is an optional amino acid sequence; B is an optional amino acid sequence; F is an optional amino acid sequence encoding an agent; G is an optional amino acid sequence encoding an agent; n is an integer of 1 or more (e.g. 2, 3, 4, 5, 6, etc.). Usually n is 1, 2 or 3. Conjugates of the invention comprising an agent that is a heterologous polypeptide comprise F and/or G. F and G can be the same agent or different agents.

For each n instances of {-X-L-}, linker amino acid sequence -L- may be present or absent. For instance, when n=2 the hybrid may be . . . X₁-L₁-X₂-L₂ . . . X₁-L₁-X₂ . . . , . . . X₁-X₂-L₂ , etc. Linker amino acid sequence(s) -L- will typically be short (e.g. 20 or fewer amino acids i.e. 20, 19, 18, 17, 16, 15, 14, 13, 12, 11, 10, 9, 8, 7, 6, 5, 4, 3, 2, 1). Examples comprise short polypeptide sequences which facilitate cloning, poly-glycine linkers (i.e. comprising Gly, where n=2, 3, 4, 5, 6, 7, 8, 9, 10 or more), and histidine tags (i.e. His where n=3, 4, 5, 6, 7, 8, 9, 10 or more). Other suitable linker amino acid sequences will be apparent to those skilled in the art. A useful linker is GSGGGG (SEQ ID NO:17) or GSGSGGGG (SEQ ID NO:18), with the Gly-Ser dipeptide being formed from a BamHI restriction site, thus aiding cloning and manipulation, and the (Gly)₄ tetrapeptide being a typical poly-glycine linker. Other suitable linkers, particularly for use as the final Lₙ are a Leu-Glu dipeptide or SEQ ID NO: 19. In some embodiments, L is absent, e.g. when X is directly attached to another X and/or B only by peptide bond.

-A- is an optional N-terminal amino acid sequence. This will typically be short (e.g. 40 or fewer amino acids i.e. 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short polypeptide sequences which facilitate cloning or purification (e.g. histidine tags i.e. His where n=3, 4, 5, 6, 7, 8, 9, 10 or more). Other suitable N-terminal amino acid sequences will be apparent to those skilled in the art. If X₁ lacks its own N-terminus methionine, -A- is preferably an oligopeptide (e.g. with 1, 2, 3, 4, 5, 6, 7 or 8 amino acids) which provides a N-terminus methionine e.g. Met-Ala-Ser, or a single Met residue.

—B— is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short polypeptide sequences which facilitate cloning or purification (e.g. comprising histidine tags i.e. His where n=3, 4, 5, 6, 7, 8, 9, 10 or more), or sequences which enhance protein stability. Other suitable C-terminal amino acid sequences will be apparent to those skilled in the art.

In some embodiments, the agent is released from the polypeptide of the invention after crossing the BBB. This may be achieved via enzymatic activity in the brain, or in response to physiochemical difference in the brain.

Producing Polypeptides of the Invention

In one embodiment, the invention provides a process for producing polypeptides and conjugates of the invention, comprising the step of culturing a host cell transformed with nucleic acid encoding a polypeptide or conjugate of the invention, under conditions which induce polypeptide expression.

The invention may use a heterologous host for expression. The heterologous host may be prokaryotic (e.g. a bacterium) or eukaryotic. It may be E. coli, but other suitable hosts include Brevibacillus chosinensis, Bacillus subtilis, Vibrio cholerae, Salmonella typhi, Salmonella typhimurium, Neisseria lactamica, Neisseria cinerea, Mycobacteria (e.g. M. tuberculosis), yeasts, etc. It is often helpful to change codons to optimise expression efficiency in such hosts without affecting the encoded amino acids.

The term “recombinant host cell” (or simply “host cell”) refers to a cell into which a recombinant expression vector has been introduced. It should be understood that such terms are intended to refer not only to the particular subject cell but to the progeny of such a cell. The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e. they are cis-acting. However, some transcriptional regulatory sequences, such as enhancers, need not be physically contiguous or located in close proximity to the coding sequences whose transcription they enhance.

The invention also provides a process for producing a polypeptide or conjugate of the invention, comprising the step of synthesising the polypeptide or conjugate chemically. Polypeptides and conjugates of the invention may be prepared by several routes, employing organic chemistry reactions, conditions, and reagents known to those skilled in the art, including: (1) reaction of a cysteine group of a modified polypeptide with a linker, to form a polypeptide -linker intermediate, via a covalent bond, followed by reaction with an activated agent; and (2) reaction of a nucleophilic group of the agent with a linker, to form an agent-linker intermediate, via a covalent bond, followed by reaction with a cysteine group of a modified polypeptide. Conjugation methods (1) and (2) may be employed with a variety of modified polypeptides, agents, and linkers to prepare the conjugates of the invention.

Pharmaceutical Compositions containing polypeptides and conjugates In another aspect, the present invention provides a composition, e.g. a pharmaceutical composition, containing one or more polypeptides and/or conjugates of the invention, formulated together with a pharmaceutically acceptable carrier, diluent or excipient. Pharmaceutical compositions of the invention can optionally be administered in combination therapy, i.e. combined with other agents. For example, the combination therapy can include a conjugate of the present invention combined with at least one other drug.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion).

Pharmaceutical compositions are preferably sterile and stable under conditions of manufacture and storage.

Compositions can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Depending on the route of administration, the polypeptide or conjugate may be coated in a material to protect it from activation, particularly after administration.

The pharmaceutical compositions of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts are known in the art.

To control tonicity, a physiological salt, such as a sodium salt, may be included. Sodium chloride (NaCl) is preferred, which may be present at between 1 and 20 mg/ml e.g. about 10±2 mg/ml NaCl. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminium hydroxide adjuvant); or a citrate buffer. Buffers will typically be included in the 5-20 mM range.

The pH of a composition will generally be between 5 and 8.1, and more typically between 6 and 8 e.g. 6.5 and 7.5, or between 7.0 and 7.8.

The composition is typically gluten free.

A pharmaceutical composition of the invention also may include a pharmaceutically acceptable antioxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

A composition may include a temperature protective agent. A liquid temperature protective agent may be added to a composition to lower its freezing point e.g. to reduce the freezing point to below 0° C. Thus the composition can be stored below 0° C., but above its freezing point, to inhibit thermal breakdown. The temperature protective agent also permits freezing of the composition while protecting mineral salt adjuvants against agglomeration or sedimentation after freezing and thawing, and may also protect the composition at elevated temperatures e.g. above 40° C. Suitable temperature protective agents should be safe for human administration, readily miscible/soluble in water, and should not damage other components in the composition. Examples include glycerin, propylene glycol, and/or polyethylene glycol (PEG). Suitable PEGs may have an average molecular weight ranging from 200-20,000 Da. In a preferred embodiment, the polyethylene glycol can have an average molecular weight of about 300 Da (TEG-300′).

Prevention of presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into the compositions.

Polypeptides and conjugates may be stabilized in formulations using combinations of different classes of excipients, e.g. (1) disaccharides (e.g. Saccharose, Trehalose) or polyols (e.g. Sorbitol, Mannitol) act as stabilizers by preferential exclusion and are also able to act as cryoprotectants during lyophilization, (2) surfactants (e.g. Polysorbat 80, Polysorbat 20) act by minimizing interactions of proteins on interfaces like liquid/ice, liquid/material-surface and/or liquid/air interfaces and (3) buffers (e.g. phosphate-, citrate-, histidine) help to control and maintain formulation pH. Accordingly, such disaccharides polyols, surfactants and buffers may be used in addition to the methods of the present invention to further stabilize polypeptides and conjugates of the invention and prevent e.g. their aggregation.

Sterile injectable solutions can be prepared by incorporating the active compound in the required amount in an appropriate solvent with one or a combination of ingredients listed herein, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of agent which can be conjugated to a polypeptide of the invention to produce a single dosage form will vary depending upon the subject being treated, and the particular mode of administration. Typically, the amount will be an amount that produces a therapeutic effect. Generally, out of one hundred per cent, this amount will range from about 0.01% to about ninety-nine% of active ingredient, preferably from about 0.1% to about 70%, most preferably from about 1% to about 30% of polypeptide or conjugate in combination with a pharmaceutically acceptable carrier.

Medical Uses and Methods of Treatment

The polypeptides and conjugates of the invention are useful in therapy. In particular, they are useful in delivering therapeutic agents to the brain. The disease to be treated will depend on the therapeutic agent that is transported by the polypeptide of the invention, but diseases of the brain are preferred. Diseases that can be treated therefore include neurological diseases, optionally a brain tumor, brain metastasis, schizophrenia, epilepsy, Alzheimer's disease, Parkinson's disease, Huntington's disease, stroke, and/or disease associated with malfunction of the BBB.

Dosages and Administration

Dosage regimens are adjusted to provide the optimum desired response (e.g. a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of polypeptide or conjugate to produce the desired therapeutic effect in association with the required pharmaceutical carrier. The specification for the dosage unit forms of the invention are dictated by and directly dependent on (a) the unique characteristics of the polypeptide or conjugate and the particular therapeutic effect to be achieved, and (b) the limitations inherent in the art of compounding such a polypeptide or conjugate for the treatment of sensitivity in individuals.

Alternatively a polypeptide or conjugate of the invention can be administered as a sustained release formulation, in which case less frequent administration is required. Dosage and frequency vary depending on the half-life of polypeptide or conjugate administered to the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the polypeptides or conjugates in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the polypeptide or conjugate (or agent) employed, the duration of the treatment, other drugs, compounds and/or materials used in combination with the polypeptide or conjugate employed, the age, sex, weight, condition, general health and prior medical history of the patient being treated, and like factors well known in the medical arts.

A “therapeutically effective dosage” of polypeptide or conjugate of the invention preferably results in a decrease in severity of disease symptoms, an increase in frequency and duration of disease symptom-free periods, or a prevention of impairment or disability due to the disease affliction.

A composition of the present invention can be administered via one or more routes of administration using one or more of a variety of methods known in the art. As will be appreciated by the skilled artisan, the route and/or mode of administration will vary depending upon the desired results. Preferred routes of administration include intracranial, intranasal, intraocular, intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, a polypeptide or conjugate of the invention can be administered via a nonparenteral route, such as a topical, epidermal or mucosal route of administration, for example, intranasally, orally, vaginally, rectally, sublingually or topically.

The polypeptides and conjugates of the invention are able to cross the Blood Brain Barrier. Accordingly, the polypeptides and conjugates are typically administered through a route that requires passage across the BBB, i.e. a peripheral administration. Commonly used administration routes that require passage across the BBB are intravenous, intramuscular, intraarterial and intraperitoneal.

The polypeptide or conjugate can be prepared with carriers that protect against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Polypeptides, conjugates or pharmaceutical compositions of the invention can be administered with medical devices known in the art.

Diagnostic Uses and Methods of Diagnosis

The polypeptides and conjugates of the invention are useful in diagnosis. In particular, they are useful in delivering diagnostic agents to the brain. The disease to be diagnosed or monitored will depend on the diagnostic agent that is transported by the polypeptide of the invention, but diseases of the brain are preferred. Diseases that can be diagnosed or monitored therefore include neurological diseases, optionally a brain tumor, brain metastasis, schizophrenia, epilepsy, Alzheimer's disease, Parkinson's disease, Huntington's disease, stroke, and/or disease associated with malfunction of the BBB.

Nucleic Acids

The invention also provides compositions comprising nucleic acids (e.g. combinations of nucleic acids, vectors, or vector combinations) encoding the polypeptides of the invention. The invention also provides compositions comprising nucleic acids (e.g. combinations of nucleic acids, vectors, or vector combinations) encoding the conjugates of the invention, particularly wherein the agent is a polypeptide.

Nucleic acids may be optimised to improve expression.

Nucleotide sequences encoding polypeptides or conjugates of the invention may be designed according to the genetic code. Thus, in the context of the present invention, such a nucleotide sequence may encode one or more of the polypeptide sequences disclosed herein.

The invention also provides nucleic acids which can hybridize to these nucleic acids. Hybridization reactions can be performed under conditions of different “stringency”. Conditions that increase stringency of a hybridization reaction of widely known and published in the art (e.g. page 7.52 of [13]). Examples of relevant conditions include (in order of increasing stringency): incubation temperatures of 25° C., 37° C., 50° C., 55° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques and their optimization are well known in the art [7, 8, 9, etc.].

A nucleic acid may hybridize to a target under low stringency conditions; in other embodiments it hybridizes under intermediate stringency conditions; in preferred embodiments, it hybridizes under high stringency conditions. An exemplary set of low stringency hybridization conditions is 50° C. and 10×SSC. An exemplary set of intermediate stringency hybridization conditions is 55° C. and 1×SSC. An exemplary set of high stringency hybridization conditions is 68° C. and 0.1×SSC.

The invention includes nucleic acid comprising sequences complementary to nucleic acid sequences encoding polypeptides or conjugates of the invention (e.g. for antisense or probing, or for use as primers).

Nucleic acids according to the invention can take various forms (e.g. single-stranded, double-stranded, vectors, primers, probes, labelled etc.). Nucleic acids of the invention may be circular or branched, but will generally be linear. Unless otherwise specified or required, any embodiment of the invention that utilizes a nucleic acid may utilize both the double-stranded form and each of two complementary single-stranded forms which make up the double-stranded form. Primers and probes are generally single-stranded, as are antisense nucleic acids.

Nucleic acids encoding polypeptides or conjugates of the invention are preferably provided in purified or substantially purified form i.e. substantially free from other nucleic acids (e.g. free from naturally-occurring nucleic acids), particularly from host cell nucleic acids, generally being at least about 50% pure (by weight), and usually at least about 90% pure.

Nucleic acids encoding polypeptides or conjugates of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. polypeptide nucleic acids (PNAs) or phosphorothioates) or modified bases. Thus the invention includes mRNA, tRNA, rRNA, ribozymes, DNA, cDNA, recombinant nucleic acids, branched nucleic acids, plasmids, vectors, probes, primers, etc. Where nucleic acid of the invention takes the form of RNA, it may or may not have a 5′ cap.

Nucleic acids encoding polypeptides or conjugates described herein may be part of a vector.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T).

Nucleic acids encoding polypeptides or conjugates of the invention can be used, for example: to produce polypeptides; as hybridization probes for the detection of nucleic acid in biological samples; to generate additional copies of the nucleic acids; to generate ribozymes or antisense oligonucleotides; as single-stranded DNA primers or probes; or as triple-strand forming oligonucleotides.

The invention provides a process for producing nucleic acid encoding polypeptides of conjugates of the invention, wherein the nucleic acid is synthesised in part or in whole using chemical means.

The invention provides vectors comprising nucleotide sequences encoding polypeptides of conjugates of the invention (e.g. cloning or expression vectors) and host cells transformed with such vectors.

As used herein, the term, “optimized” means that a nucleotide sequence has been altered to encode an amino acid sequence using codons that are preferred in the production cell or organism, generally a eukaryotic cell, for example, a cell of Pichia, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence. Optimized expression of these sequences in other eukaryotic cells is also envisioned herein. The amino acid sequences encoded by optimized nucleotide sequences are also referred to as optimized.

Sequence Identity For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. When comparing two sequences for identity, it is not necessary that the sequences be contiguous, but any gap would carry with it a penalty that would reduce the overall percent identity. For blastn, the default parameters are Gap opening penalty=5 and Gap extension penalty=2. For blastp, the default parameters are Gap opening penalty=11 and Gap extension penalty=1.

Percent sequence identities referred to herein are determined in accordance with BLAST algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands.

General

The term “amino acid sequence”, as it is used in this document, should be taken to include reference to each of the sequences disclosed herein, as well as to their fragments, homologues, derivatives and variants.

The term “comprising” encompasses “including” as well as “consisting” e.g. a composition “comprising” X may consist exclusively of X or may include something additional e.g. X+Y.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The term “about” in relation to a numerical value x means x±10%.

The practice of the present invention will employ, unless otherwise indicated, conventional methods of chemistry, biochemistry, molecular biology, immunology and pharmacology, within the skill of the art. Such techniques are explained fully in the literature. See [10-17].

The invention is further described with reference to the following non-limiting examples.

### EXAMPLES

Compilation of the LA-Interacting Polypeptide Database

To compile an LA-interacting polypeptide database, the inventors gathered structural information on all polypeptides known to be able to interact with the LA domain of the LDLR family members.

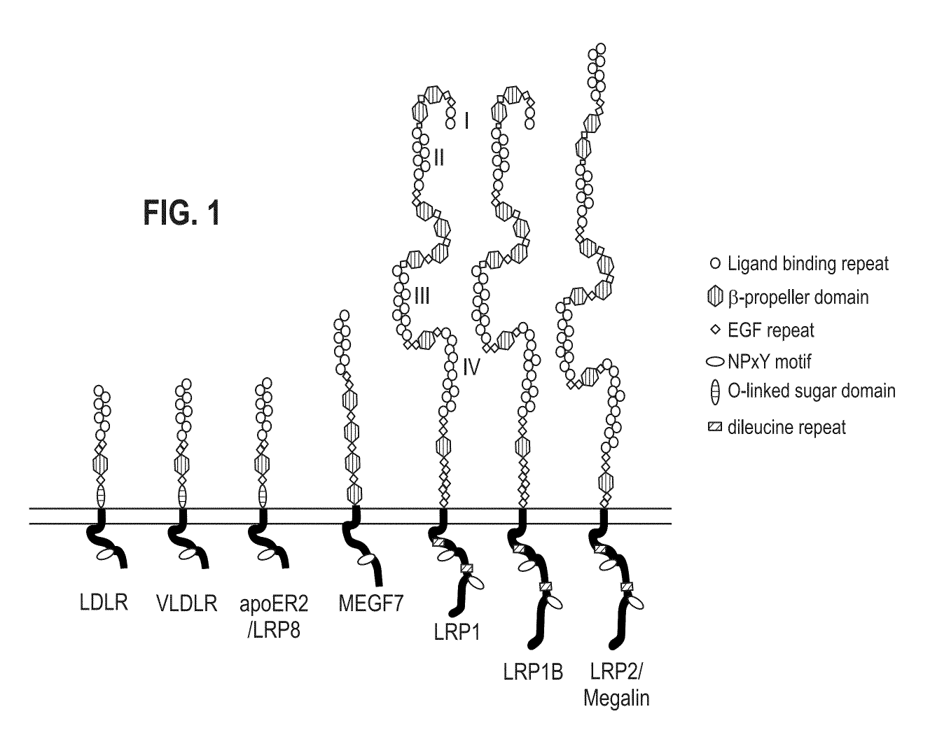

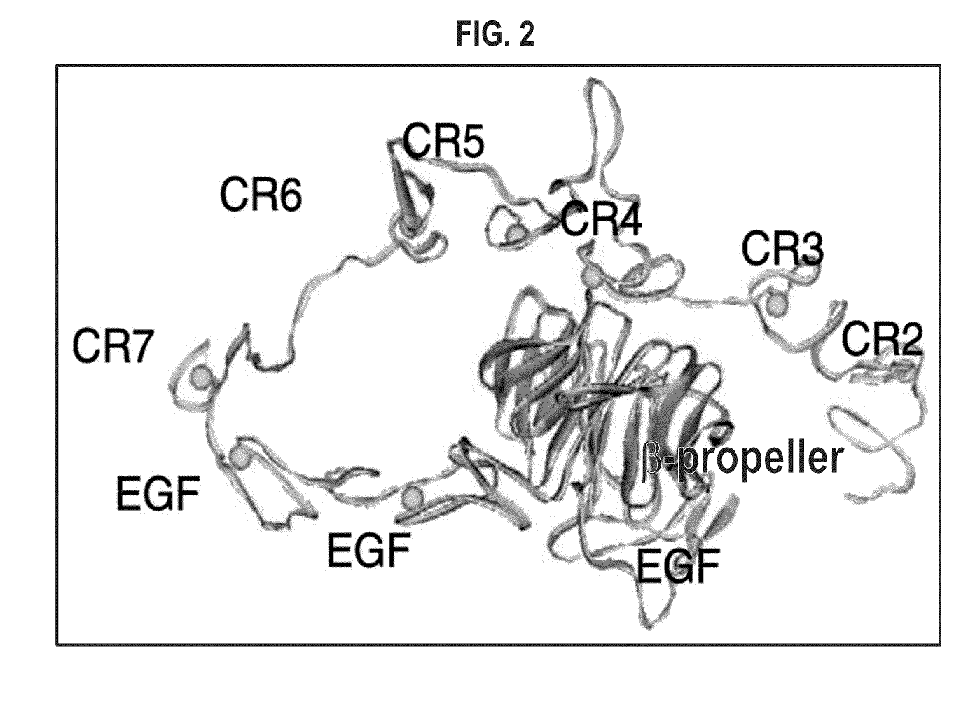

These structural data allowed the inventors to deduce important features that a chemical entity must have to interact with the LA domain. The following LDLR family members were considered (see FIG. 1):
1. LDLR (low density lipoprotein receptor) is the prototype of all family members;
2. VLDLR (very low density lipoprotein receptor);
3. ApoER2 (apolipoprotein E-receptor 2, also known as LRP8);
4. MEGF7 (Epidermal growth factor-like protein 7, also known as LRP4);
5. LRP1 (LDL receptor-related proteins 1, also known as LRP);
6. LRP1B (LDL receptor-related protein 1B)
7. LRP2 (LDL receptor-related proteins 2, also known as Megalin).

TABLE 1
Interaction partners of LDLR-family LA domain [references 18, 19, 20, 21, 22, 23, 24, 25,
26, 27, 28, 29, 30 and 31].

| Host(module/ | | | | |
|---|---|---|---|---|
| domain) | Ligand(section) | Method used in the study | Comments | References |
| LRP1(LA) | Angiopeps | Transcytosys and volume of | | Demeule et al. |
| | | distribution measures. | | |
| LRP1(LA16-18) | apoE(130-149) | NMR | Fusion protein between host | Guttman et al. |
| | | | and ligand. | |
| | | | Activity data available. | |
| | | | PDB code: 2KNY | |
| LRP1(sLRPs) | 1) apoE(130-149) | Solution binding assays. | Double domain binder (ligand)*. | Croy et al. |
| | 2) apoE(141-155)² | Surface plasmon resonance | Affinity data available. | |
| LDLR(LA4-LA5) | RAP (D3) | Crystallography | PDB code: 2FCW | Fischer et al. |
| | | | Double domain binder (ligand). | |
| LDLR(LA4-LA5) | LDLR(YWTD, β-propeller) | Crystallography | Intramolecular interaction. | Rudenko et al. |
| | | | PDB code: 1N7D. | |
| | | | Double domain binder (ligand)*. | |
| VLDLR(V3) | HRV(VP1) | Crystallography | PDB code: 1V9U | Verdaguer et al. |
| | | | Single domain binder (ligand)*. | |
| LDLR(LA4) | B2GPI(D5) | NMR relaxation, NMR titration, | PDB code: 2KRI | Lee et al. |
| | | molecular docking guided by | Single domain binder (ligand)*. | Beglov et al. |
| | | NMR-derived restraints and | Haddock model. | Related studies: |
| | | extensively validated (Haddock) | Activity data range estimated. | Pennings et al. |
| ApoER2(LA1) | Reelin(repeat 5-6) | Crystallography | PDB code: 3A7O | Yasui et al. |
| | | | Single domain binder (ligand)*. | |
| | | | Activity data available. | |
| ApoER2(LA4) | B2GPI(D5) | Mutation studies, | Accordng to Lee et al.16 it is | Van Lummel et al. |
| | | immunoprecipitation. | very unlikely that the Lysines | |
| | | | from the loop form the main | |
| | | | contact site in the B2GPI(D5)- | |
| | | | LA complex. | |
| LRP1(LA5_LA6) | RAP(D2) | NMR, NMR-derived restraints | Haddock model | Jensen et al. |
| | | docking (Haddock) | PDB code: 2FYL | |
| | | | Double domain binder (ligand)*. | |
| | | | Activity data available. | |
| LDLR(LA7) | LDLR(EGF(A)) | NMR | Intramolecular interaction | Beglova et al. |
| | | | Single domain binder (ligand)*. | |
| | | PDB code: 1XFE | Different folding of LDLR | |
| | | | respect the others seen. | |

*Single or double binder stand for the number of LA modules with which the ligand interacts simultaneously.

In Table 1 we report all the binding partners (ligands) of each LDLR family member, which are found in the literature. The group of ligands that interact with LA modules of LDLR members are named herein as the “LA-interacting polypeptide database”.

Rationalization of Important Interacting Patterns

The information found for the interacting partners of the LDLR family LA domain can be divided into 2 parts: one part relates to the activity data and the other relates to structural information. In the literature, few activity data sets were obtained using comparable experimental conditions, and so the use of these data for the building of predictive models is not generally recommended. Nevertheless, the inventors were able to extract and exploit certain important information from these structural data.

The inventors found that these structural data are very heterogeneous:
Some ligands bind to just a single LA domain, while some others establish interactions with 2 modules (taking advantage of an avidity effect).
Some ligands (e.g. the small angiopep polypeptides) interact with residues which are localized close to each other in respect to the primary structure, while others interact with residues that are very far away from each other, and constitute a different part of the amino acid sequence.
Some ligands interact with flexible structure regions (such as loops), while others interact with rigid ones (such as a-helices).

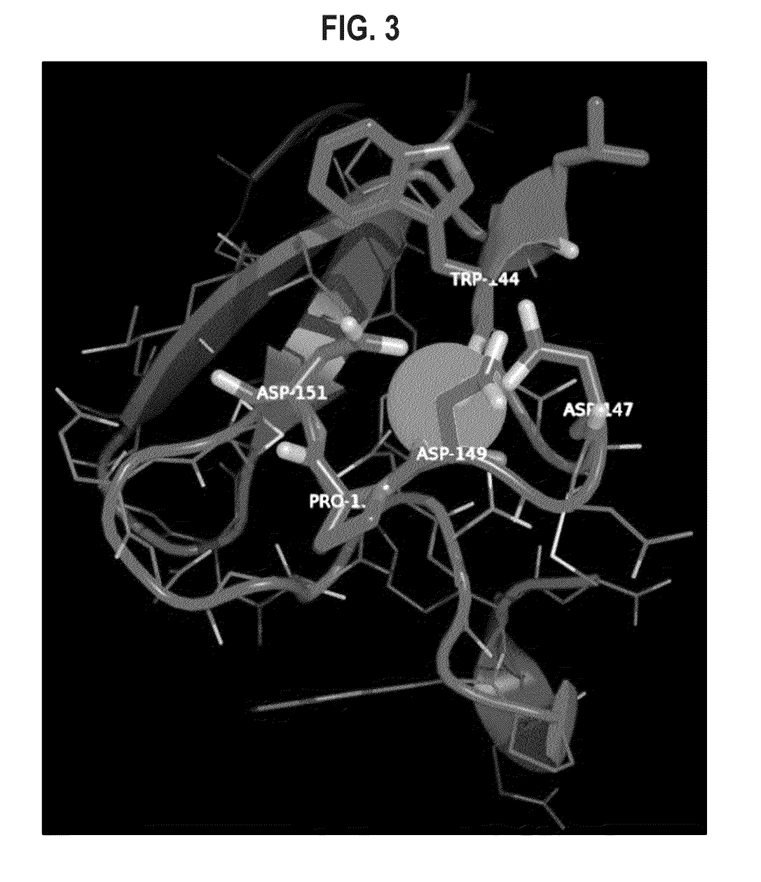

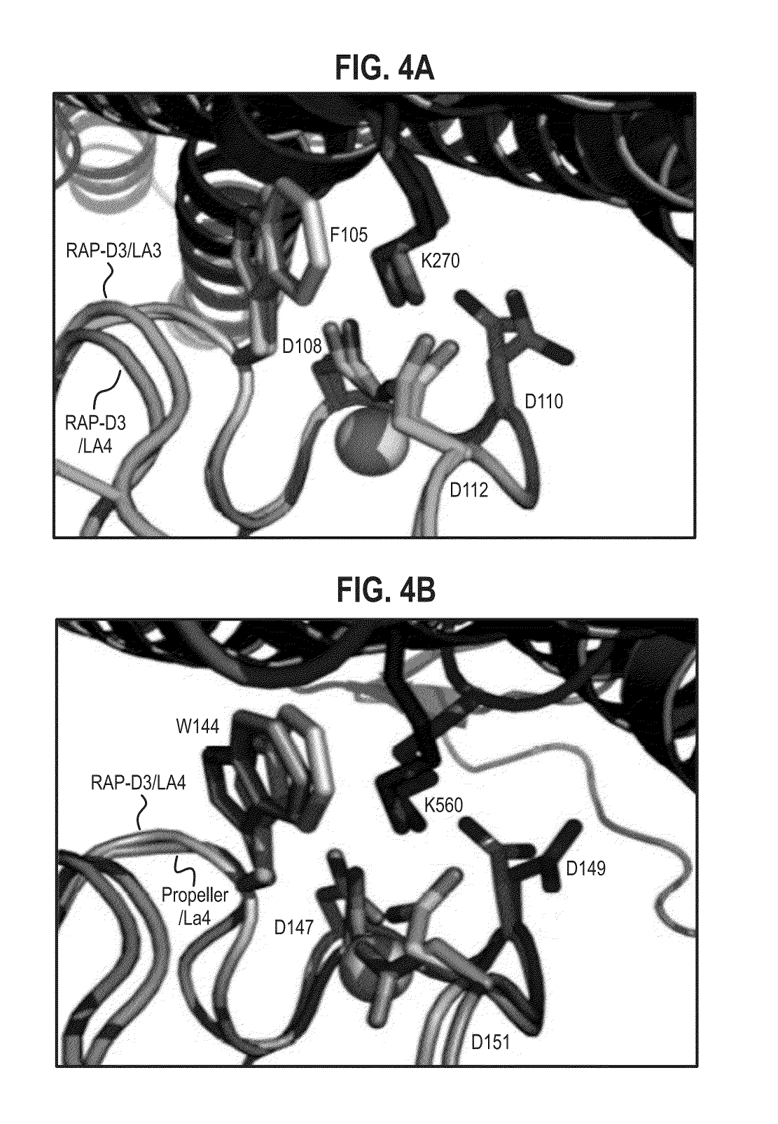

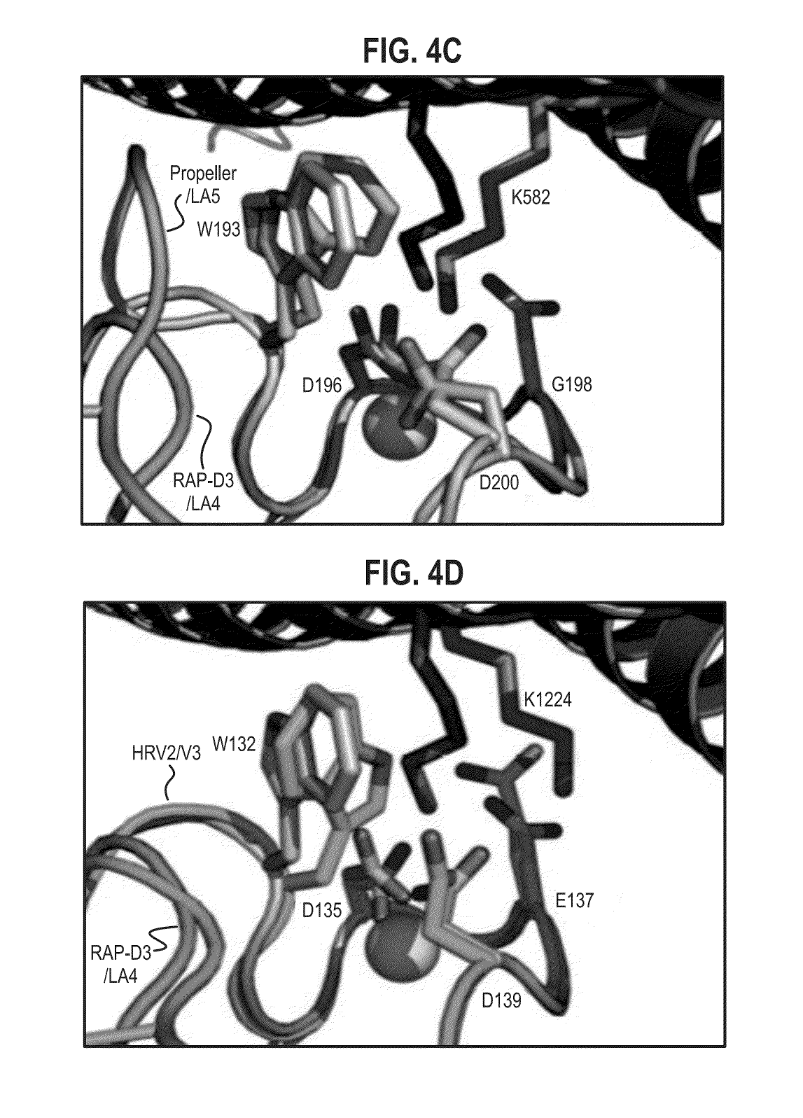

While the ligands in the LA-interacting polypeptide database were found to be very heterogeneous, the inventors found that, surprisingly, folding of all LA modules of all the LDLR family members are highly conserved. For example, the inventors found that the LA4 module of the complex between 2 LA domains of LDLR and the receptor associated protein (RAP) co-crystalised, as described by Fisher and co-workers [21] (see FIG. 3). Each LA module has a short (3-hairpin near the N-terminal end, 3 disulfide bonds, and a highly conserved calcium binding site. The calcium ion is coordinated in an octahedral geometry by the side chain of 4 acidic residues, which are highly conserved (D147, D151, D157, E158), and 2 backbone carbonyl groups (W144 and D149) [32]. All these acidic groups, together with an aromatic residue (W144 on LA4 and F105 on LA3) are universally present in all LRP1 LA module pairs which bind with high affinity to RAP [33]. In all structures of the studied complexes, the essential element of binding resides in a strong electrostatic interaction, where multiple H-bonds are formed between a positively charged Lysine (ligand) and the conserved acidic groups, forming a negatively charged crown around the calcium ion (receptor). Moreover, the conserved aromatic residue in the receptor stabilizes with hydrophobic interactions in the un-polar region of the Lysine chain, as shown for several interacting protein partners in the Blacklow review [34] (see FIG. 4).

The complete list of all the ligand residues forming H-bonds or hydrophobic interactions with LA domains is shown in Table 2. Structures which have 2 table entries have 2 interaction domains. (e.g.: 2FCW represents the interaction between RAP and the LA4 domain of LDLR, while 2FCW2 stands for the interaction between RAP and the LA3 domain of the same receptor).

TABLE 2
Residues of ligands forming H-bonds or hydrophobic interactions with LA domains. Residues
with superscript *are the essential lysines complexed with the highly conserved acidic crown system of
LA modules. Residues with superscript **are long chain H-bond donor residues. Residues with
superscript \hat{ }form hydrophobic interactions with W144 of the receptor. Residues with superscript ^{!}form
hydrophobic interaction with L143. Residues with superscript ^{#}form hydrophobic interactions with
P150. Hydrophobic interactions marked with “(b)” involve only backbone atoms.

| Feature | 2FCW2 | 2FCW2 | 1V9U | 2KRI | 2KNY | 3A7Q | 2FYL | 2FYL2 | 1N7D |
|---|---|---|---|---|---|---|---|---|---|
| H-bonding | (NH3)** | (NH3)* | (NH3)* | (NH3)** | (NH) | (O)I2348 | (NH3) | (O)Q27 | (COO) |
| residues | K253** | K270* | K224* | K282** | S129 | (NH3)* | K24 | (COO) | E581 |
| | (NH3)* | (NH2)** | (NH) | (O) | (NH) | K2467* | (NH3)* | E30 | (NH3)* |
| | K256* | R285** | N88 | K284 | E131 | (NH3)** | K60* | (NH3) | K582* |
| | (OH) | (NH3)** | (NH) | (NH3)* | (NH2) | K2360** | (COO) | K31 | |
| | Y260 | K289** | N90 | K308* | R134 | | D65 | (NH2) | |
| | (NH2) | | | (n-H)** | (O)S139 | | (COO) | R34 | |
| | R296 | | | H310** | (NH2) | | E74 | (O)G92 | |
| | | | | (n-H) | R142 | | | (NH3)* | |
| | | | | W316 | (NH3)* | | | K93* | |
| | | | | (NH3) | K143* | | | | |
| | | | | K317 | (NH3) | | | | |
| | | | | | K146 | | | | |
| | | | | | (NH) | | | | |
| | | | | | K147 | | | | |
| Hydrophobic | A252^{!} | I266^{!} | N88(b) | K282 | T130 | M2345^{!} | M22 | Q27 | H562 |
| interaction | K253 | E269 | Y89 | K284 | E131 | I2553\hat{ } | K24 | E30 | E581\hat{ } |
| residues | [symbol] \hat{ } | [symbol] \hat{ } | A87 | K305(b)^{!} | R134 | E2359^{!} | L57 | K31 | [symbol] 582\hat{ } |
| | [symbol] \hat{ } | R282 | [symbol] \hat{ } | C306(b)^{!} | V135 | K2360 | K60 | R34 | |
| | Y260 | R285^{#} | [symbol] \hat{ } | K308 | A138^{!} | V2389\hat{ } | K63\hat{ } | K93 | |
| | R296^{#} | K289 | I266^{!} | H310 | S139 | K2467 | L64 | | |
| | L300 | | | W316^{#} | L141 | Q2468 | D65 | | |
| | | | | K317 | [symbol] \hat{ } | I2470\hat{ } | E74 | | |
| | | | | [symbol] \hat{ } | [symbol] \hat{ } | | | | |
| | | | | | [symbol] \hat{ } | | | | |
| | | | | | R147 | | | | |
| | | | | | L148 | | | | |

[symbol]

indicates data missing or illegible when filed

In Table 3 we schematize the mostly conserved pharmacophoric features observed.

TABLE 3
Mostly conserved pharmacophoric features. *These residues are slightly displaced in
respect to those of reference 2FCW.

| Feature | 2FCW | 2FCW2 | 1V9U | 2KRI | 2KNY | 3A7Q | 2FYL | 2FYL2 | 1N7D |
|---|---|---|---|---|---|---|---|---|---|
| | K256 | K270 | K224 | K308 | K143 | K2467 | — | — | K582 |
| | K253 | K289* | A87(b) | K282 | | K2360 | — | — | — |
| | | | N88(b) | | | | | | |
| | | | N90(b) | | | | | | |
| | R296 | R285 | — | W316 | — | — | — | — | — |
| | Y260 | — | — | K317 | — | — | — | — | — |
| | | | | H310* | | | | | |
| | A252 | I266 | L86 | — | A138 | I2358 | — | — | — |
| | | | I226 | | | M2345* | | | |

2.3 Modeling of Known Polypeptides Able to Cross the BBB

In addition to the information gathered from the interacting polypeptide database, the inventors also analysed polypeptides already known to be able to cross the BBB. In particular, the inventors chose to study the angiopeps and regulon. No structural information is available for these polypeptides, and so the inventors generated models to work with these polypeptides.

The angiopeps, introduced in 2007 by Demeule and co-workers [1,2] are the only polypeptides known to date to be able to cross the BBB with a mechanism involving the LDLR. The regulon polypeptide is known to cross the BBB.

Angiopeps

Aprotinin is a 6500-Da protease inhibitor ligand of LRP and LRP2 containing a Kunitz-type domain. The angiopeps is a family of polypeptides derived from the Kunitz domain that show a higher transcytosis capacity than aprotinin. In Table 4 the inventors report transcytosis and volume of distribution values for different angiopeps. The inventors identified angiopep-2 (AP2) as the most promising polypeptide because it has both a good transcytosis level and a high Parenchyma/Total brain volume of distribution ratio.

A first analysis of these polypeptides was performed considering, for each of its constituting AAs, 113 physicochemical properties reported by Cai and co-workers [35]. The polypeptides are then clustered using an agglomerative hierarchical clustering method in the following manner:
Dissimilarity between each AA is calculated as the normalized euclidean distance in the 113 dimensions space.
The distance between each polypeptide is calculated as the sum of the distances between AA that are in the same position.

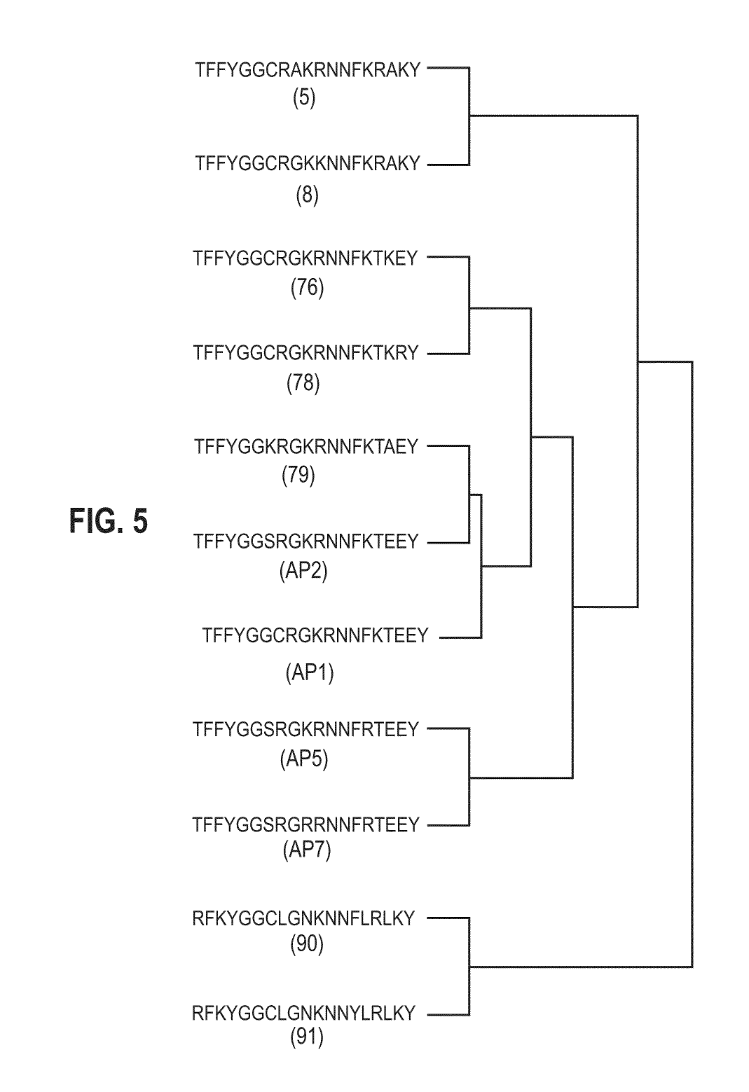

The clustering results are shown in FIG. 5. The inventors found that these polypeptides can be divided into 3 main groups: (A) angipeps 5 and 8; (B) angiopeps 76, 78, 79, AP2, AP1, AP5, AP7; and (C) angiopeps 90 and 91. This clustering, obtained using only AA physicochemical properties, reflects significant changes in transcytosis: group (A) has a low percentage of transcytosis; group (B) has moderate values (but for API. Data for AP5 and AP7 are missing); group (C) has the highest value for transcytosis.

TABLE 4
Volume of distribution for Angiopeps polypeptides.

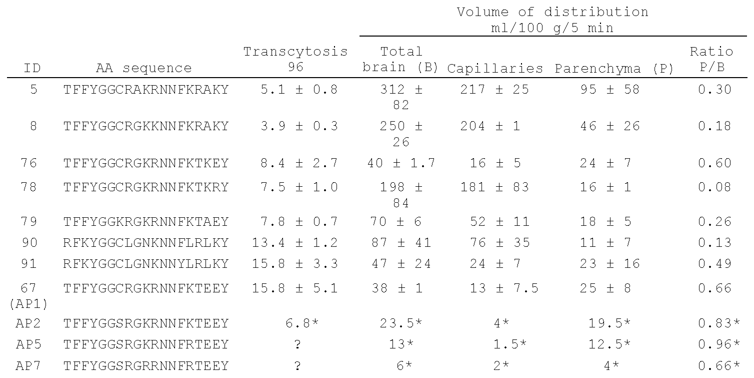

*These values were extrapolated from the original publication graphs.

Further insights into the effect of different amino acids on transcytosis is traditionally gained from structural analyses, but there is currently no structural information available for angiopeps. Therefore, the inventors built a hypothetical structure for the angiopeps, following a homology model strategy using the crystallized Kunitz domain of aprotinin as the template (PDB code 2ZJX). The alignment with angiopep AP2 is shown below, which only misses the last amino acid (Y) due to the functioning of the homology modelling program:

| | | | |
|---|---|---|---|
| AP2 | 1 | TFFYGGSRGKRNNFKTEE | 18 |
| 2ZJX_A | 32 | TFVYGGARAKRNNFKSAE | 49 |

The inventors selected aprotinin as the template for the following reasons:
A sequence identity of 72% is obtained, which is well above the common threshold of 35% for homology modelling.
Both lysines (which are potentially essential for the interaction with the LDLR) are present.
Residues that are essential in the binding to LDLR are potentially well oriented and exposed.

The general fold of AP2 obtained by homology modelling is shown in FIG. 6.

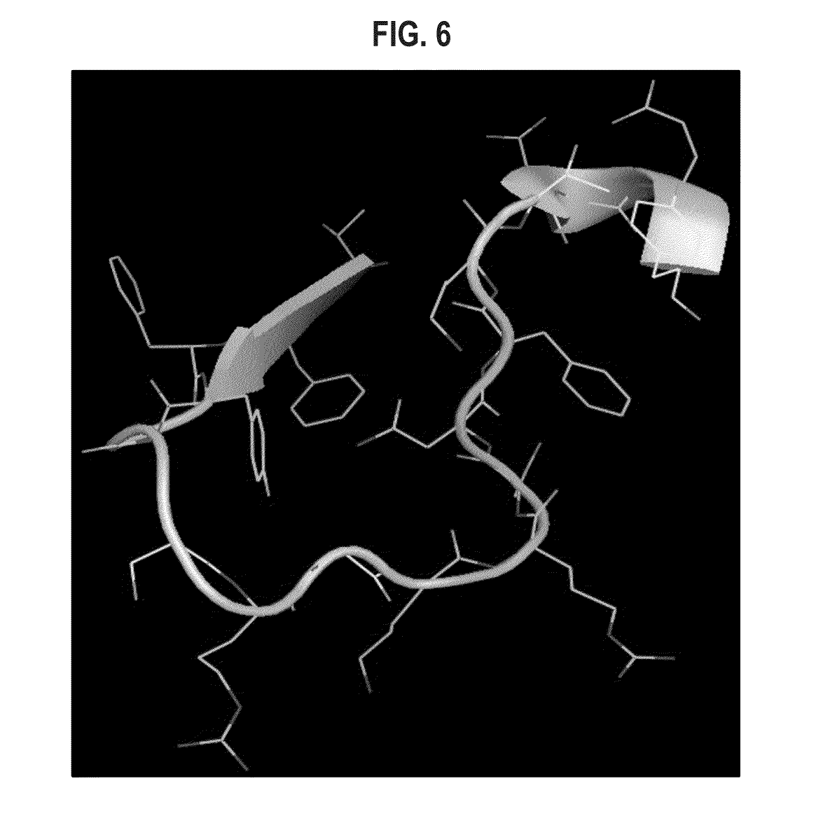

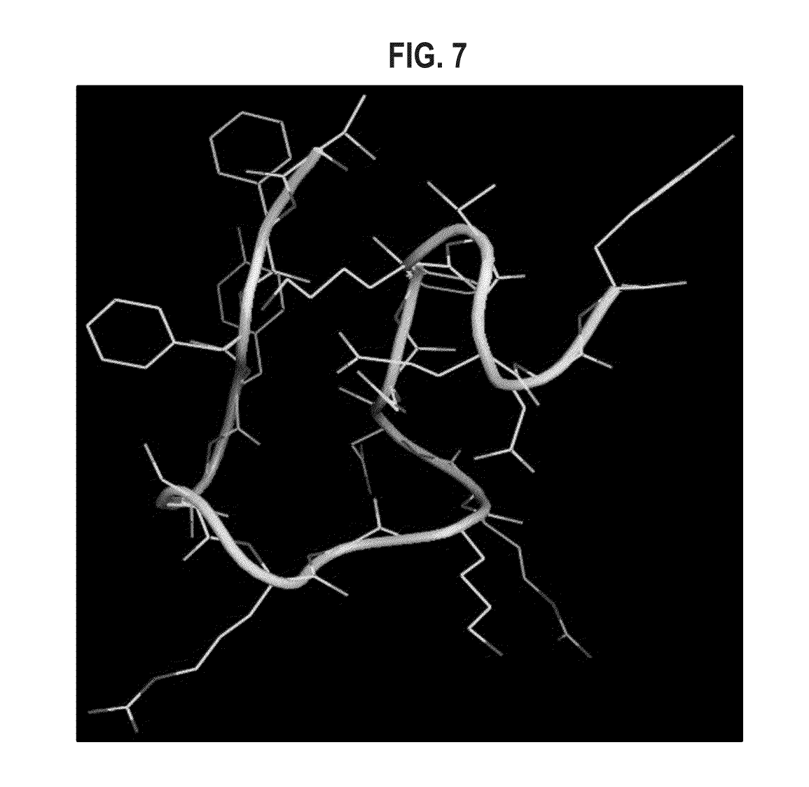

An ab-initio method [36,37,38] for fold prediction was used to confirm the homology model of AP2. The inventors found that this ab-initio model gives a similar fold as the homology model (see FIG. 7).

Other than AP2, other angiopeps also show Kunitz-like folds when using homology modelling. Based on these results, the inventors propose that angiopeps have Kunitz-like folds.

Modelling AP2-LDLR Interaction

The inventors then modelled the interactions between AP2 and LDLR. Two main hypotheses were considered: (A) AP2 as a single domain binder of LDLR (i.e. that it binds to a single LA domain, as observed for complexes: 2FCW, 1N7D, 2FYL); and (B) AP2 as double domain binder (i.e. that it binds to 2 LA domains, as observed for: 2KNY, 1V9U, 2KRY, 3A7Q). All alignments were performed using the LDLR-RAP complex structure (PDB 2FCW) as the template. The inventors consider this the best structure for several reasons: (i) it is a crystallographic structure; (ii) it has a good resolution (1.26 A); and (iii) the number of interactions between the ligand and the receptor are higher than in other experimental structures.

AP2 as Single Domain Binder

The homology structure of AP2 was rigidly (only translational and rotational movements) aligned with the RAP key residues (see Table 3) of RAP-LDLR crystallographic complex in several ways. Model_{—}1, which maximizes the number of superposed equivalent residues (Table 5), is considered to be the most appropriate:

TABLE 5
Superposition of AP2 homology model to RAP-LDLR
complex (PDB code 2FCW).

| | Aligned with: | | | | |
|---|---|---|---|---|---|
| | K256 | K253 | R29 | Y260 | Eval- |
| Model | (2FCW) | (2FCW) | (2FCW) | (2FCW) | uation |
| model_1 | K10 | R11 | R8 | not far | 5 |
| | | | | from Y4 | |
| model_2 | K10 | — | — | R11 | 1 |
| model_3 | K10 | R8 | — | — | 1 |
| model_4 | K15 | — | R11 | | 3 |
| model_5 | K15 | — | — | Y19 | 2 |
| model_6 * | K15 | — | — | — | 4 |

Evaluation range: 1(bad)-5(very good).

Model_{—}1 optimizes the superposition of 3 key residues of RAP-LDLR interaction: K256, which is the essential interacting lysine, K253 and R296 which act as H-bonds donors of the conserved acidic residues of LDLR. (* This construct was built by docking the AP2 as double domain binder, using AP2 K15 over RAP K256 and AP2 K10 over RAP K270 (equivalent to K256 in another LA module)).

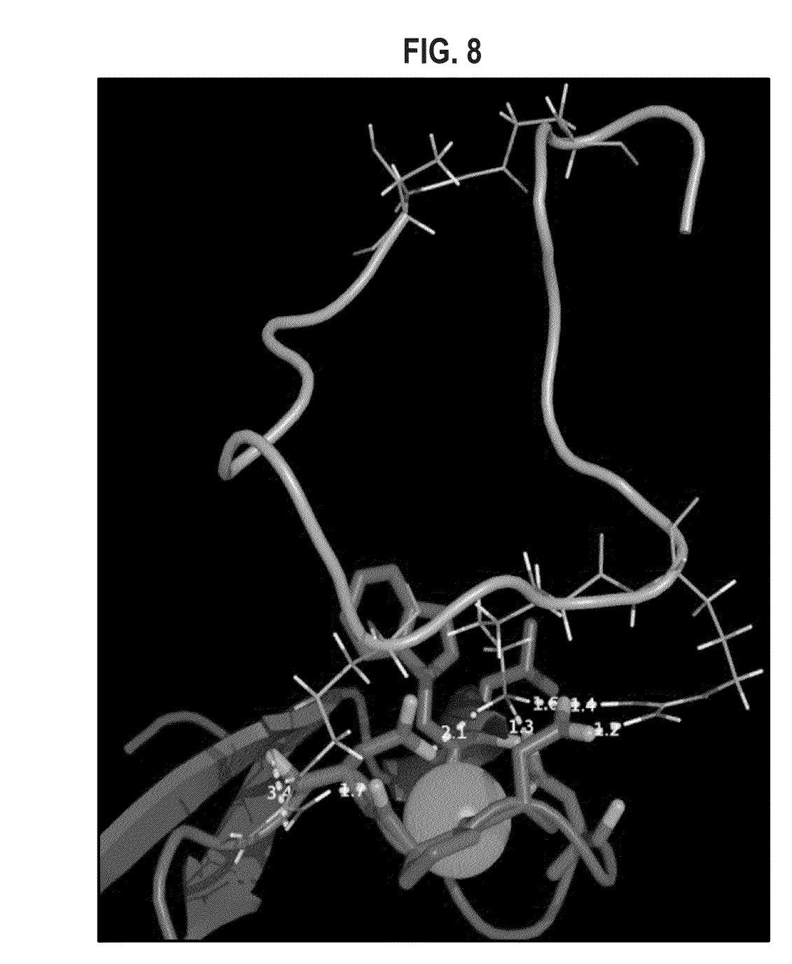

A minimization of Model_{—}1 is done with the force field MMFF94 [39]. The final minimized structure is shown in FIG. 8.

AP2 as Double Domain Binder

Several experimental observations have shown that some polypeptides bind to a pair of LA modules rather than to a single module [22,33]. A rational explanation of this is that an avidity effect plays a major role in the binding of ligands to LDLR family proteins [21]. In the crystal structure solved by Fisher and co-workers, the contact interface between each individual LA module and RAP is small (<400 A2), so they suggest that a single module would be insufficient to provide high-affinity binding.

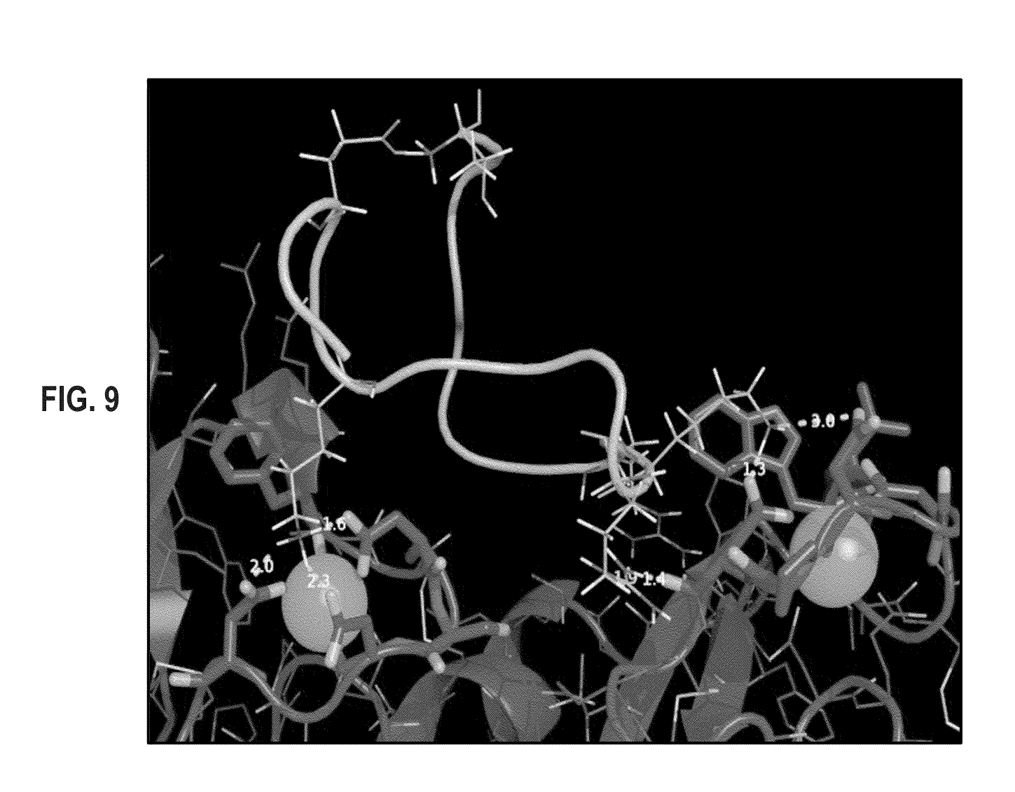

For these reasons, the inventors also considered whether AP2 is a double domain binder. AP2 was aligned with RAP by superimposing K15 (AP2) with K256 (RAP) and K10 (AP2) with K270 (RAP) (model_{—}6 of Table 5). The model is minimized using the MMFF94 [39] force field, giving the final structure shown in FIG. 9.

AP2 Interacting Features

In both models (AP2 as single and double domain binder) 3 important residues (R8, K10 and R11) interacted strongly with one LA module, forming H-bonds with the acidic receptor residues conserved in all LDLR family members. Additionally in model_{—}6 (AP2 double domain binder), K15 forms a H-bond with the crown acidic residues of the other LA module. All these residues (lysines and arginines) are present in several known ligands of LA domains (Table 2).

The inventors found that an intramolecular H-bond may be established between the N-terminal residue of the polypeptide and the side chain of E17. This interaction could restrict the flexibility of the polypeptide and promote the AP2-LA interaction. This could explain the much lower P/B volume distribution ratio of angiopep-79 in respect to AP2. These 2 polypeptides are indeed very similar but angiopep-79 lacks the glutamic acid two amino acids away from the C-terminus (see FIG. 5). Similarly, the inventors found that all the polypeptides having a P/B volume distribution ratio larger than 0.50 have an acidic residue in the C terminal end. The only exception is angiopep-91 which has a P/B ratio =0.49. Other relationships between the predicted Angiopep-2 structure and changes in amino acids which cause a change in BBB penetration could not be detected.

In conclusion, the inventors provided a new structural model for the angiopeps. 5 residues were identified as being important for the interaction with the LA modules, and a further residue for the stabilization of the fold of the angiopeps.

With these new data, the inventors developed new polypeptides that bind to the LDLR and cross the BBB. These polypeptides fall into 2 main groups (1) some are based on the regulon sequence and are expected to have a beta-hairpin fold; (2) others are based on RAP, which interacts with LDLR.

Regulon

Objectives

To analyze the crossing of our nanoparticles with and without Regulon peptide (SEQ ID No.1) through an in vitro model of BBB (blood brain barrier).

Materials and Methods

The BBB Model

To provide an in vitro system for studying brain capillary functions, the inventors have developed a process of co-culture that closely mimics the in vivo BBB by culturing brain capillary endothelial cells on one side of an insert and glial cells (astrocytes) on the other. Endothelial cells are cultured in the upper compartment on the filter and astrocytes cells in the lower compartment on the plastic of a six-wells plate.

Under these conditions, endothelial cells retain the endothelial markers (factor VIII-related antigen, non thrombogenic surface, production of prostacyclin, angiotensin-converting enzyme activity) and the characteristics of the BBB (presence of tight junctions, paucity of pinocytotic vesicles etc.).
Astrocytes Culture

Primary cultures of fresh astrocytes were provided from Innoprot (ref. P10202). They were maintained 48 h in the initial flask. Cells were seeded in AM-a culture medium (Innoprot, ref.1831) in a plate P100. When the confluence was 80-90%, astrocytes were seeded in a 6-wells plate (125.000 cells/well (2 ml)). The co-culture was established after 48-72 h.
Bovine Brain Microvascular Endothelial Cells (BBMVECs Cells)

Endothelial cells from Cell Applications Inc. were frozen in nitrogen. Cells were defrosted and seeded in medium for BBMVECs (Cell Applications) in a plate P100 after a process of “coating” (Attachment factor solution from Cell Applications 30 min 37° C.+1 μg/ml fibronectine from Sigma 10 min 37° C.). When the cellular confluence was 60-70%, cells were seeded in inserts (1.0 μm pore for a 6-well plate, Millipore, PIRP3OR48) after a process of coating (150000-200000 cells for insert). The co-culture was established after 48-72 h.
Establishment of the BBB Model

The astrocytes medium was aspirated, and BBMVECs medium was added in the wells with astrocytes. The inserts were transferred to the wells with the astrocytes. Then we had the endothelial cells in the upper compartment (luminal compartment) and the astrocytes in the lower compartment (abluminal compartment).
Measure of TEER (Transepithelial Electrical Resistance)

The resistance was measured between the luminal and abluminal compartments after 72 h from the co-culture establishment (each measure per triplicate)

A good BBB model (in which the tight junctions are formed) is considered where the TEER values are above 150 Ω×cm².

Transcytosis Experiment

A transcytosis experiment was carried out in 2 BBB models with TEER values of 230 Ω×cm², testing nanoparticles decorated with the Regulon peptide (SEQ ID No. 1) and nanoparticles that are not decorated with the Regulon peptide, and one insert blank without cells (for NP non-decorated).

The samples were prepared: 250 μg NP/ml in Ringer's solution. All the samples were sonicated for 10 min. 1.5 ml of samples was added in the inserts, and 2.5 ml of Ringer solution in the well (6-wells plate).

Results

After 60 min at 37° C. in agitation, the samples were collected in the upper and lower compartment, and analyzed with an NTA analysis.

| | blank | NPs decorated | NPs not decorated |
|---|---|---|---|
| Up | 3.42 × 10⁸ particles/ml | 3.05 × 10⁸ | 3.94 × 10⁸ particles/ |
| | | particles/ml | ml |
| down | 3.61 × 10⁸ particles/ml | 2.9 × 10⁸ | 0.61 × 10⁸ particles/ |
| | | particles/ml | ml |
| % | 105.555556 | 95.0819672 | 15.4822335 |
| crossing | | | |

These data are shown graphically in FIG. 23.

Conclusion

In a good BBB model (TEER 230 Ω×cm²), very few of the non-decorated nanoparticles crossed the barrier, while through the blank (insert without BBB), all the non-decorated particles cross the barrier.

In the case of the nanoparticles decorated with the peptide, almost all the nanoparticles (95%) cross the BBB. So the Regulon peptide has an important role in the crossing mechanism through the BBB.

Regulon Constructs

Regulon is able to cross the BBB. As noted above, using co-culture of bovine brain endothelial capillary cells and rat primary astrocytes as an in vitro BBB model, the inventors found that nanoparticles decorated with regulon can cross the BBB, whereas uncoated nanoparticles do not cross the BBB. A negative control did not cross the BBB, confirming that the BBB was kept intact while the decorated nanoparticles crossed it. These experimental data are shown in FIG. 23. Accordingly, FIG. 23 shows that Regulon (SEQ ID No. 1) is able to transport nanoparticles across the BBB.

Regulon is made up of 59 AAs (SEQ ID NO:1), and so is larger than the angiopeps (that contain 19 AAs). No sequence similarity is encountered between angiopeps and regulon (according to BLAST comparison method). Furthermore, no structural information about regulon is known. To investigate the possible transcytosis of regulon by the LDLR, the inventors performed a structural study, allowing creation of a homology model of Regulon.

The inventors found the best template for homology modeling of regulon to be the P62 envelope glycoprotein (PDB code 3N40_P), which has a high sequence identity of 68%, as shown below:

| | | | |
|---|---|---|---|
| | | 10 20 30 40 50 60 | |
| | | ....*....\|....*....\|....*....\|....*....\|....*....\|....*....\| | |
| Regulon | 1 | HKKWQFNSPFVPRADEPA-RKGKVHIPFPLDNITCRVPMAREPTVIHGKREVTLHLHPDH | 59 |
| 3N40_P | 292 | HKKWQYNSPLVPRNAELGdRKGKIHIPFPLANVTCRVPKARNPTVTYGKNQVIMLLYPDH | 351 |

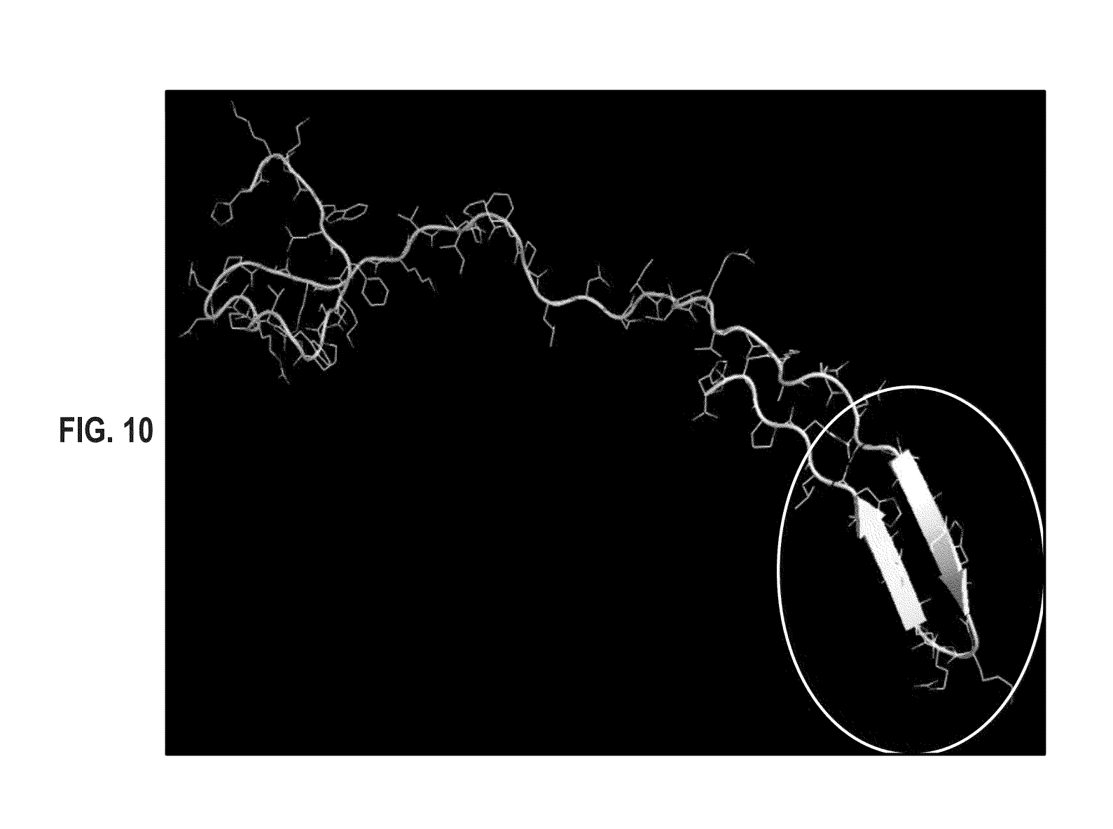

The general fold of regulon homology model is shown in FIG. 10. The homology model structure obtained by the inventors can be divided into 2 parts: (1) a rigid β-hairpin structure (circled in FIGS. 10); and (2) a long unstructured flexible chain.

The inventors note that exactly at the U-turn of the n-hairpin are 2 residues (K48 and R49) that are essential for a potential interaction with LDLR.

Modelling Regulon-LDLR Interaction

As for AP2, the inventors modelled the interactions between regulon and LDLR. In this case the inventors note that there is still no available evidence relating the interaction between regulon and LDLR LA-module. Again, two main hypotheses were considered: (1) regulon as a single domain binder of LDLR; and (2) as a double domain binder. All alignments were performed using the homology model of regulon and the LDLR-RAP complex structure (PDB 2FCW) as the template.

Regulon as Single Domain Binder

The primary hypothesis for the binding of regulon with the LA module is that the polypeptide interacts with its structured part (circled in FIG. 10). As mentioned above, this area corresponds to a β-hairpin (β-strand, U-turn, β-strand), and has 2 AAs in the U-turn that are essential for the binding of other molecules to a LA module (lysine and arginine). The side-chains of these 2 residues are exposed and well oriented for a possible interaction. For this reason we create the model overlapping the (1) regulon U-turn lysine (K48) with the essential interacting lysine of RAP (K256) and (2) the U-turn arginine (R49) with 2 different residues of RAP: R296 (model_{—}1); and K253 (model_{—}2).

Both models were positively evaluated because both K48 and R49 form H-bonds with the acidic conserved residues of the LA module (see Table 6).

TABLE 6
Superposition of regulon homology model to RAP-LDLR
complex (PDB code 2FCW).

| | Aligned with: | | | | |
|---|---|---|---|---|---|
| | K256 | K253 | R296 | Y26 | |
| Model | (2FCW) | (2FCW) | (2FCW) | (2FCW) | Evaluation |
| model_1 | K48 | — | R49 | — | 5 |
| model_2 | K48 | R49 | — | — | 5 |

Evaluation range: 1(bad)-5(very good).

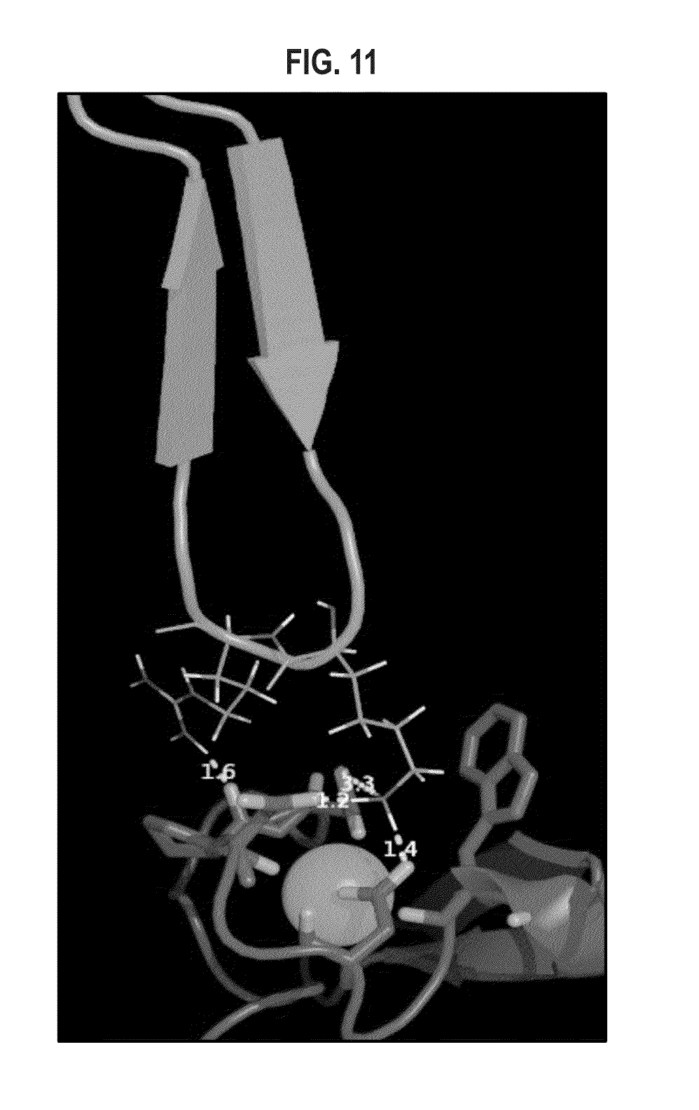

Even though the interaction of regulon K48 and R49 with important key residues of the LA module is very strong, and structurally fits well, the inventors believe that the interaction of these 2 residues by themselves is insufficient for the binding of the whole regulon (59 residues). This hypotheis is based on the interacting surface area (FIG. 11), which is rather small with respect to the total regulon surface. The inventors believe that the flexible loop of regulon participates in the binding, and so regulon is considered as a double domain binder.

Regulon as Double Domain Binder

Treating regulon as a rigid structure, it cannot interact simultaneously with 2 LA modules in the same fashion as AP2. The template does not allow the two essential lysines be at a correct distance to establish a double binder model. To model possible alternative regulon-LA module interaction patterns, the inventors “cut” regulon into different pieces, to build subset models with those.

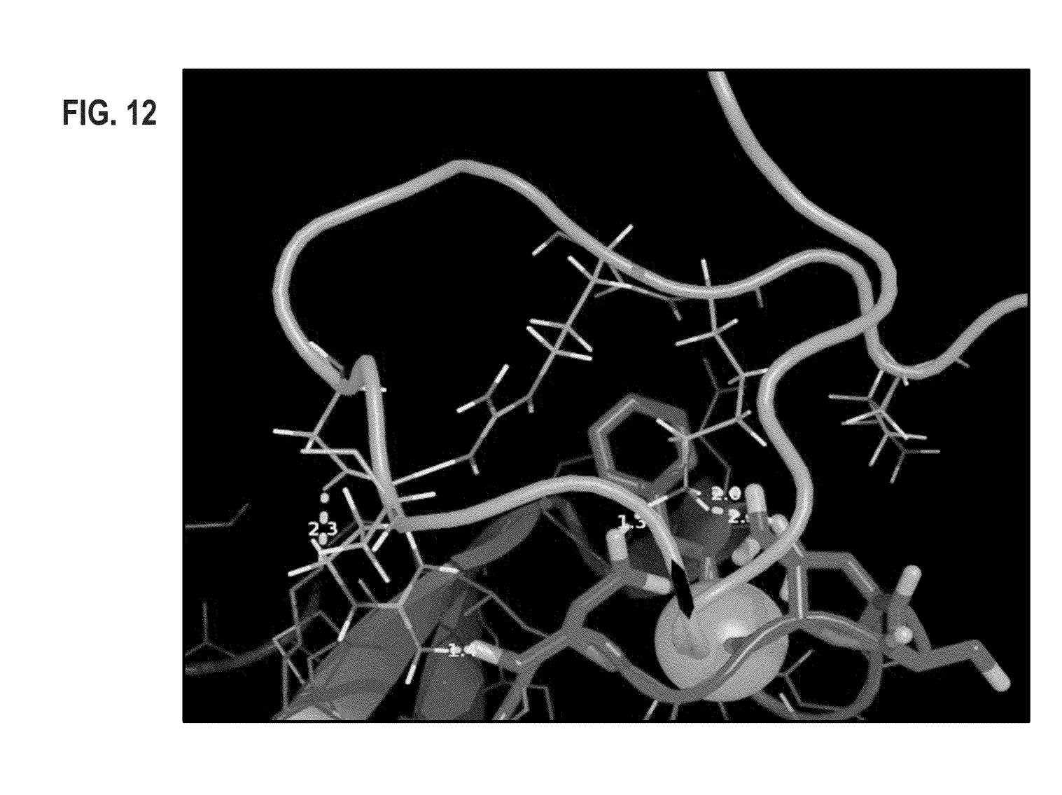

Apart from the sequence including K48 & R49 (as mentioned above), the inventors identified 2 other main areas that can interact (always based on lysine/arginine patterns) with the LA module: one includes the K2- K3 motif, and the other includes the residues R13, R19 and K20, K22. The most favourable of these is the latter, because it has a larger interacting surface than the former. Moreover, it also has a residue composition and configuration that resembles the flexible Kunitz domain of AP2. As before, the superimposition was carried out by a superimposition with the LDLR-RAP complex, followed by a minimization of the system. In the minimized structure, the following regulon residues establish H-bonds with the LA module: R13, D15, K20 (see FIG. 12). When molecular dynamics simulations are applied, R19 and K22 start to form H-bonds with LA module.

Based on the flexibility of regulon, the inventors propose that it acts as a two LA module binder, by using the hairpin region as well as the part spanning from residue R13 to K20.

Description of the Regulon Structure

From the homology modelling approach described above, the inventors propose that regulon's structure is a long flexible loop, where the only structured part is a beta-hairpin localized near the C-terminal region (FIG. 10). The hairpin is formed by the following residues:

| | |
|---|---|
| TVIHGKREVTLH | (SEQ ID NO: 20) |

The beta-hairpin is made from 2 beta-strands and a U-turn. The 2 beta-strands are localized in the following regions:

| | |
|---|---|
| TVIHG | (SEQ ID NO: 21) |
| EVTLH | (SEQ ID NO: 22) |

The U-turn is made from the 2 essential AAs:
KR

Shorter Regulon Sequences

The inventors thus propose that regulon interacts with the LDLR using its hairpin region. The inventors designed two new polypeptides for use in crossing the BBB, which include the C-terminal part of regulon. 2 constructs were designed:

Regulon_construct1
(SEQ ID NO: 2)
Sequence: PTVIHGKREVTLHL
Length: 14 AAs.

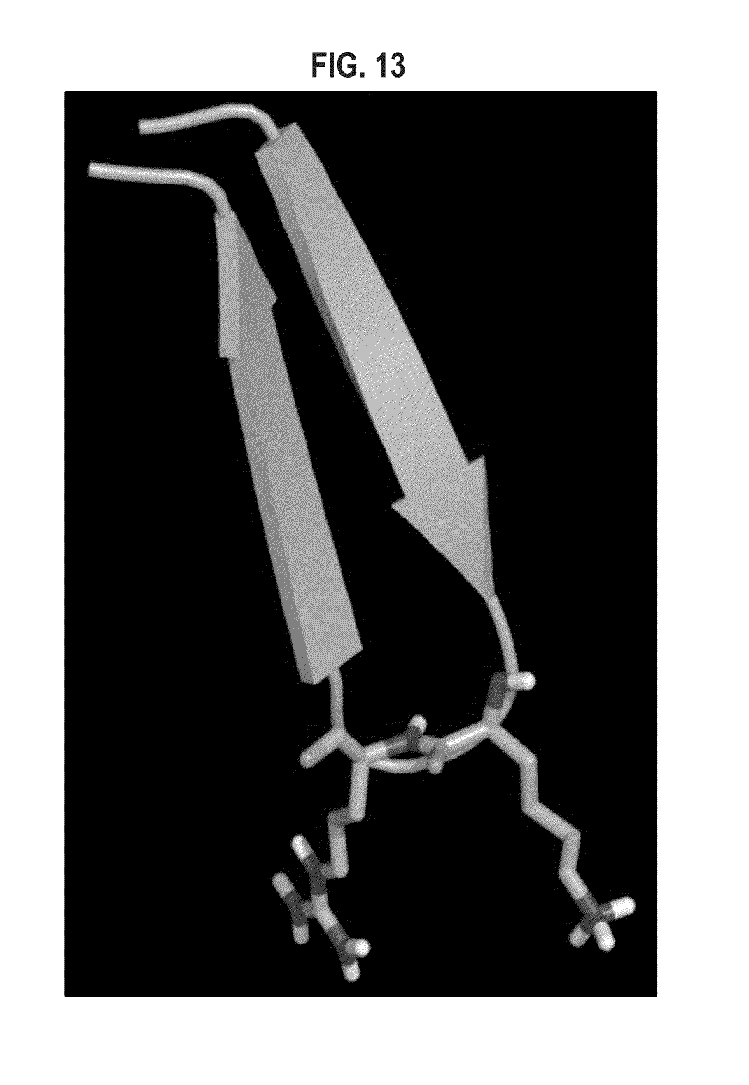

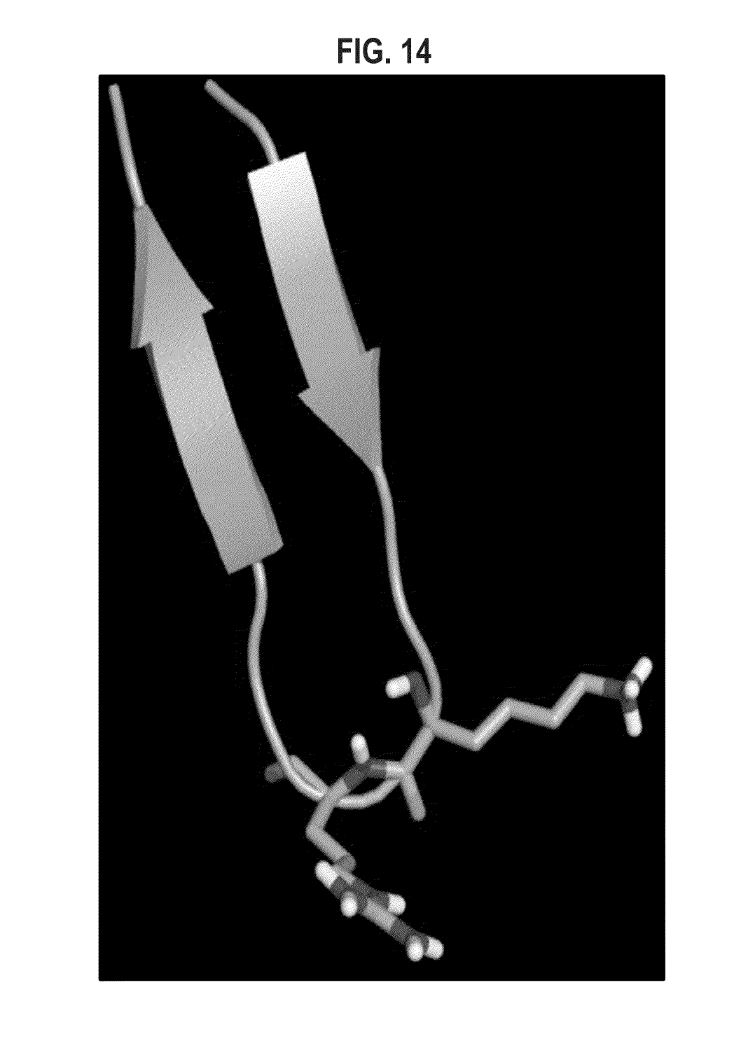

The “Regulon_construct1” polypeptide includes the minimal structured region of regulon. Its structure includes only the beta-hairpin area (see FIG. 13). To validate the conservation of a beta-hairpin in this shorter polypeptide, the inventors used an ab-initio method to predict the structure of regulon_construct1. A beta hairpin structure was observed (see FIG. 14).

Regulon_construct4
(SEQ ID NO: 3)
Sequence: PMAREPTVIHGKREVTLHLHPDH
Length: 23 AAs.

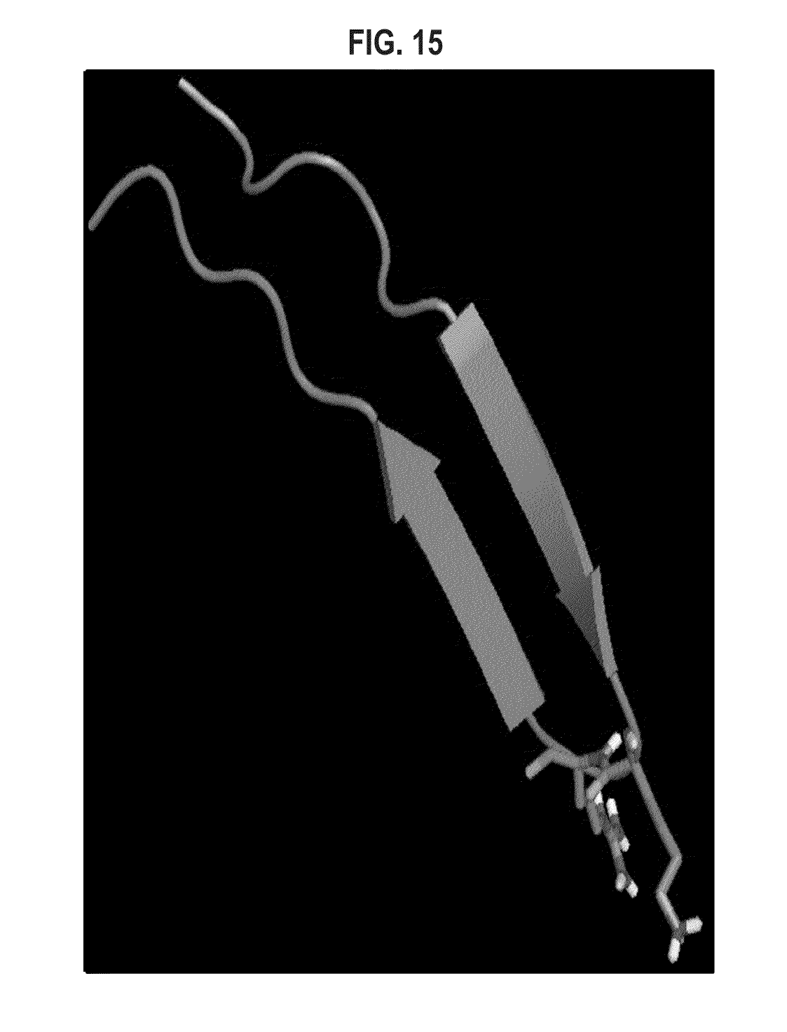

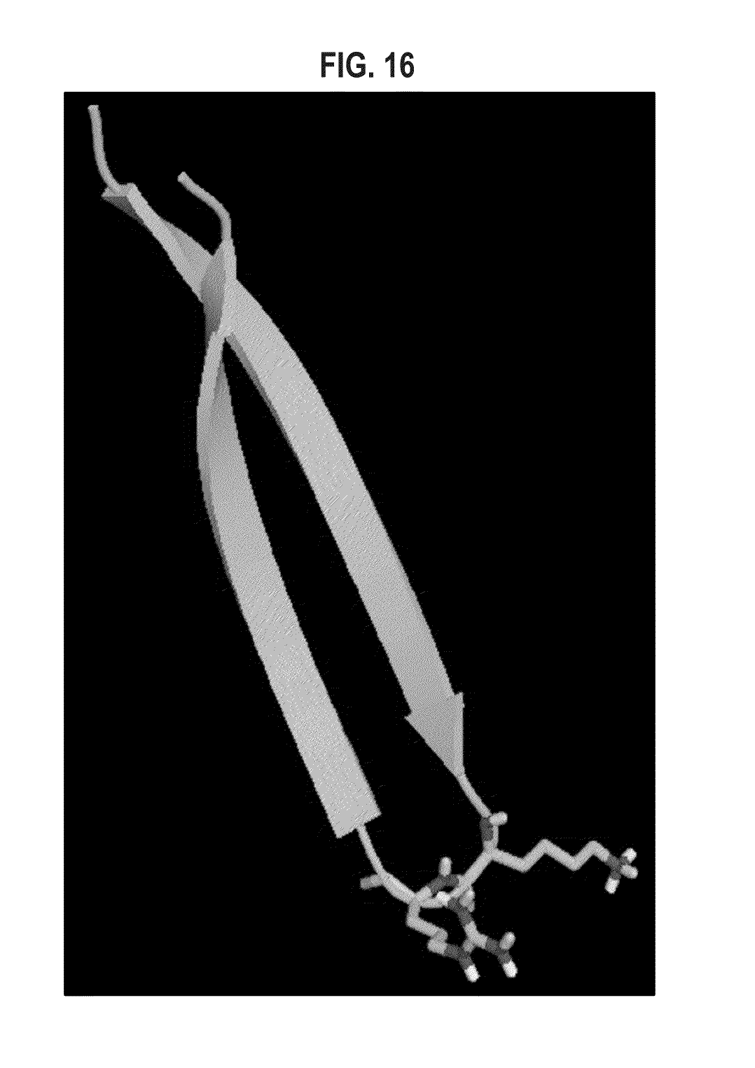

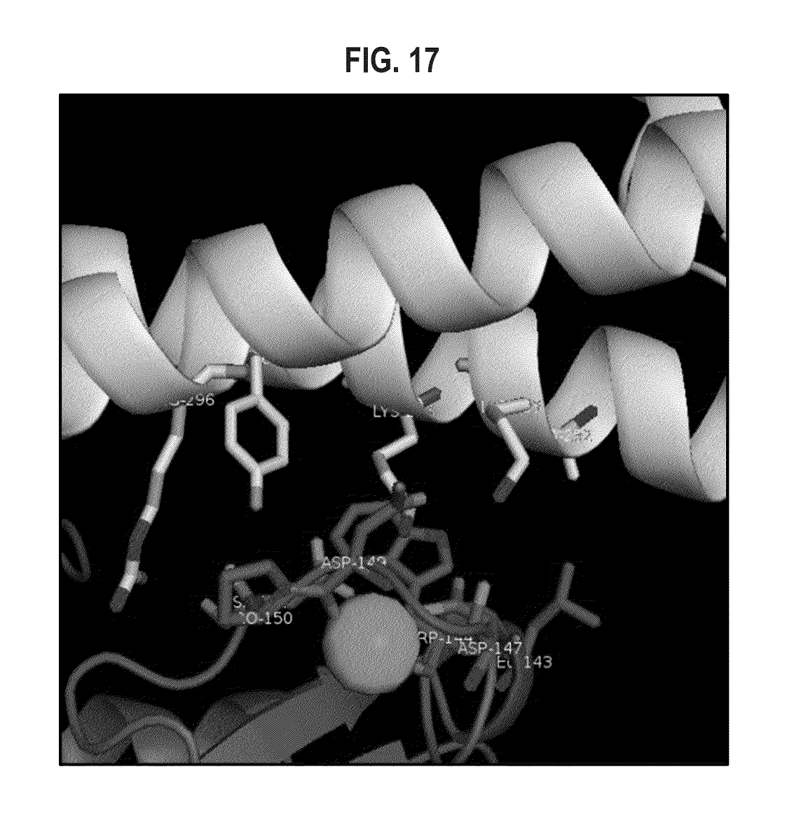

The “Regulon_construct4” polypeptide includes the whole sequence of regulon_construct1 and additional sequences on both ends of the beta-hairpin. All amino acids of the C-terminal of regulon are included. The inventors propose that regulon_construct4 is a particularly suitable polypeptide because the additional sequences represent parallel loops that can, in theory, form a beta-sheet. (see FIG. 15). The inventors used an ab initio method to predict the structure of regulon_construct4 and obtained an elongated beta hairpin structure for the whole polypeptide (see FIG. 16).

RAP Alpha-Helix

As mentioned above, the D3 domain of RAP is known to be able to bind to LDLR. The interaction takes place through 2 alpha-helices of RAP with 2 LDL receptor type-A (LA) modules. A single alpha-helix contains the essential residues for the interaction, including LYS256, while the other one seems to stabilize the complex. The inventors designed 3 new polypeptides based on the RAP-D3 sequence.

RH_construct1
(SEQ ID NO: 4)
Sequence: ELKHFEAKIEKHNHYQKQLE
Length: 20 AAs.

The RH_construct1 polypeptide was identified by the inventors as the minimal unit of RAP-D3 interacting with LDLR (see FIG. 18). The inventors used an ab-initio method to predict the structure of RH_construct1 and observed a fully alpha-helix structure from end to end (see FIG. 19).

RH_construct2
(SEQ ID NO: 5)
Sequence:
DKELEAFREELKHFEAKIEKHNHYQKQLEIAHEKLRHAESV
Length: 41 AAs.

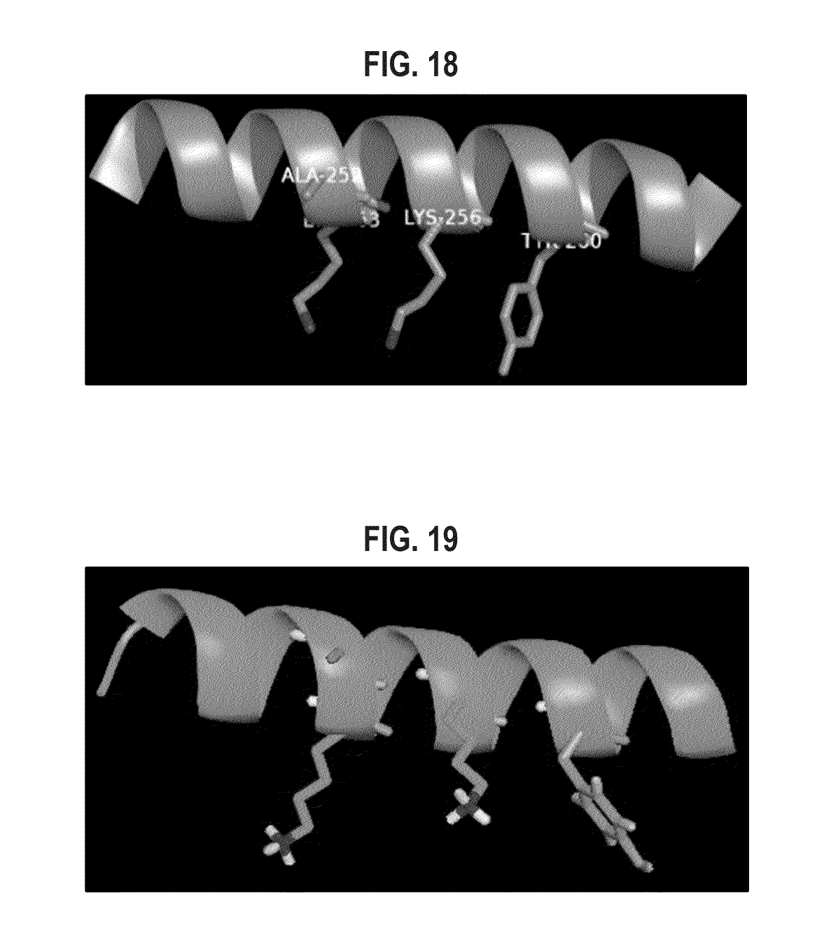

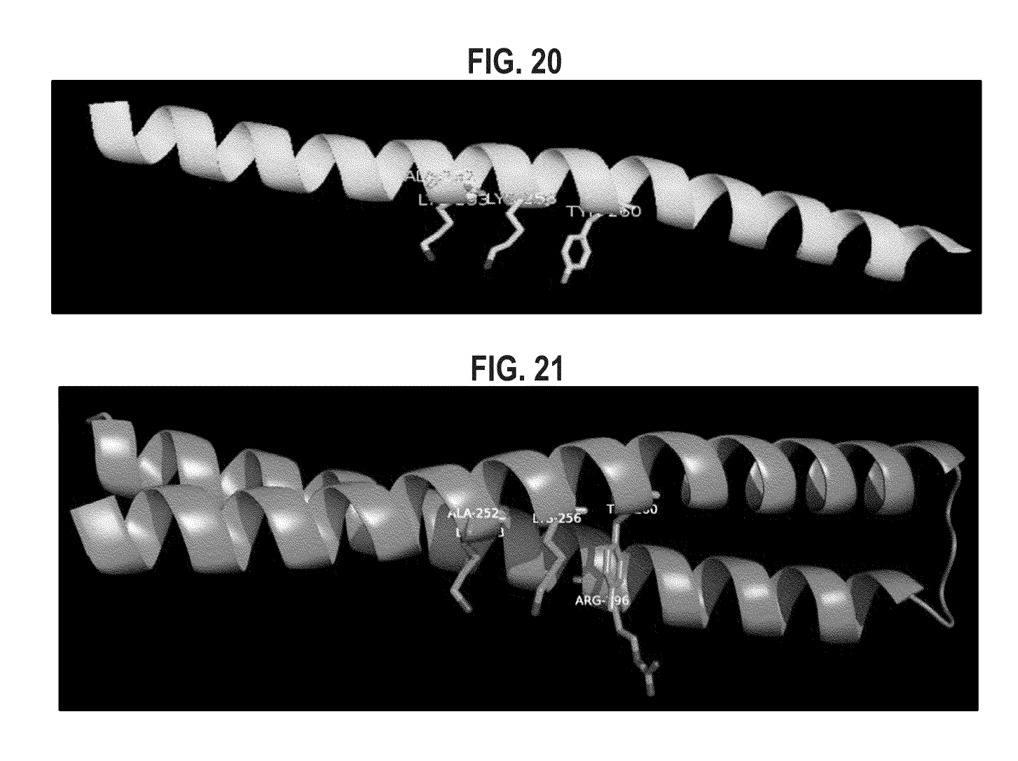

The RH_construct2 polypeptide identified to the inventors corresponds to the full alpha-helix of RAP including the essential residues for the interaction with LDLR. Here the inventors took a longer segment in respect to RH_construct1 to favour the formation of α-helix, and include additional regions to assist the translocation of the BBB. The ab-initio prediction method could not be used for this polypeptide because it is optimized for polypeptides with a length of 9 to 30 residues, and the RH_construct2 has 41 residues. To confirm the stability of the α-helix, the inventors performed a short molecular dynamics simulation of 100 ps. According to the simulation, the α-helix is relatively stable (see FIG. 20).

RH_construct3
(SEQ ID NO: 6)
Sequence:
DKELEAFREELKHFEAKIEKHNHYQKQLEIAHEKLRHAESVGDGERVSRS
REKHALLEGRTKELGYTVKKHLQDLSGRISRARH
Length: 84 AAs.

The RH_construct3 polypeptide, including most of the RAP D3 domain, is formed by 2 α-helices. In addition to the α-helix of the RH_construct2 polypeptide, including the residues essentials for the interaction with LDLR, this construct is made up of a second α-helix containing Arg296 that interacts with LDLR to stabilize the complex.

Again, the ab-initio prediction method could not be applied to RH_construct3 because it contains more than 30 residues. To confirm the stability of the α-helices, the inventors performed a short molecular dynamics simulation of 100 ps. According to the simulation, the a-helices appear to be stable (see FIG. 21).

Design of Further New Polypeptides

The inventors designed further new polypeptides that differ from the AP2 and RAP template sequences.

From the compiled interacting polypeptide database (Table 2), the inventors distinguished 2 types of interactions with the LDLR members:
1. Some polypeptides interact with the LDLR using residues located in different flexible areas (e.g.: 1V9U, 2KRI, 3A7Q, 1N7D).
2. Other polypeptides interact with LDLR using residues located in a-helices (e.g.: 2FCW, 2FCW2, 2KNY, 2FYL, 2FYL2).

The inventors designed polypeptides for both interaction types: referred to herein as “flexible” and “rigid” polypeptides.

Flexible Polypeptides Design of flexible polypeptides that are able to bind to the LA module of LDLR was based mainly on the important interacting features identified in the AP2 double binder model. The following AP2 residues were kept fixed in the inventors' models, because they are considered to function in promoting the interaction with LA domain: R8, G9, K10, R11, K15, E17.

All of the “flexible” polypeptides have the following design criteria:
Essential AP2 residues are kept to maintain the interaction with LA module (R8, G9, K10, R11, K15, E17).
AP2 non-essential residues are modified in order to:
   maximize the intermolecular interactions
   maximize the intramolecular interactions
   form a polypeptide with high propensity to adopt a flexible-loop secondary structure
   form a polypeptide with high probability to be soluble in water

The AA secondary structure propensity was established following the criteria determined by Costantini and co-workers [40], which is an improved version of the popular Chou-Fasman method [41,42].

The list of flexible polypeptides designed by the inventors is presented in Table 7.

TABLE 7
Flexible polypeptides for the binding of the LA module. The highlighted amino acids are those
conserved from the AP2 double binder model. Flex 1 is SEQ ID NO: 7; flex 2 is SEQ ID NO: 8; flex 3 is
SEQ ID NO: 9; flex 4 is SEQ ID NO: 10; flex 5 is SEQ ID NO: 11.

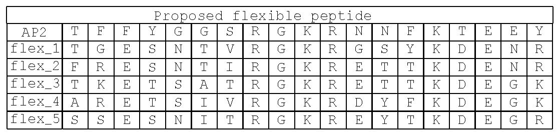

As an example, the inventors detail the design of all flex_{—}1 residues:
T1 was chosen to promote an hydrophobic intramolecular interaction with Y14 to promote the doubled Kunitz-type folding.
G2 was chosen to be a residue with high propensity to form a flexible-loop.
E3 was chosen to promote a H-bonding intermolecular interaction with R103 of LA module.
S4 was chosen to be a polar residue with high propensity to form a flexible-loop.
N5 was chosen to be a polar residue with high propensity to form a flexible-loop.
T6 was chosen to promote a hydrophobic intermolecular interaction with V106 of the LA module.
V7 was chosen to promote a hydrophobic intermolecular interaction with T126 of the LA module.
R8 was a AP2 residue considered essential for the interaction with LA module.
G9 was a AP2 residue considered essential for the interaction with LA module.
K10 was a AP2 residue considered essential for the interaction with LA module.
R11 was a AP2 residue considered essential for the interaction with LA module.
G12 was chosen to be a residue with high propensity to form a flexible-loop.
S13 was chosen to be a polar residue with high propensity to form a flexible-loop.
Y14 was chosen to promote a hydrophobic intramolecular interaction with T1 to promote the doubled Kunitz-type folding.
K15 was a AP2 residue considered essential for the interaction with LA module.
D16 was chosen to promote an H-bonding intermolecular interaction with Q104 of LA module.
E17 was a AP2 residue considered essential to interact intramolecularly with N-end and promote the doubled Kunitz-type folding.
N18 was chosen to be a polar residue with high propensity to form a flexible-loop.
R19 was chosen to be a polar residue to form an H-bonding intermolecular interaction with D110 of LA module.

Rigid Polypeptides

The inventors based their “rigid” polypeptide design strategy on the co-crystallized RAP LA complex, instead of AP2. RAP is a large polypeptide of 106 AAs forming 3 connected rigid α-helices. RAP interacts with 2 LA modules using residues from its 2 largest α-helices.

The inventors designed a smaller polypeptide which forms a single α-helix, which can act as a single or as a double domain binder. The main interacting motif of RAP was identified as “K**K***Y”, with the second lysine being the essential one. The other 2 residues play the role of H-bond donor groups for important carboxylate and carbonyl moieties of the receptor. Thus, the inventors propose that the first lysine and the tyrosine residues may be exchanged for lysine or arginine residues, which are also H-bond donors and positively charged, allowing them to interact with acidic receptor residues in an optimal manner. The other residues of the RAP mimetic should be AAs with a high propensity to form α-helices.

Alias and co-workers successfully used the pattern of “Ac-YGDAAAE-X-EAAAAG-NH2” to obtain an α-helical polypeptide [43]. The multi-alanine motif assures the formation of an α-helix structure, and the aspartate residues are proposed to increase the polypeptide's solubility and the tyrosine residue to facilitate spectroscopic quantification of polypeptide concentration [44].

The double domain binders designed by the inventors are larger than the polypeptide of Alias and co-workers, and even though multi-alanine residues assure formation of an α-helix structure, alanine is a nonpolar residue which causes solubility problems. To circumvent such solubility problems, the inventors chose to mutate the alanine residues to glutamate residues on the opposite side of the binding surface of the α helix. Glutamate was chosen due to its negative charge, thereby increasing polypeptide solubility and potentially orientating the polypeptide's positively charged side (which is the binding site of ligand) toward the negatively charged surface of the receptor. Moreover, glutamate residues have a high propensity to form α-helices. Finally, residues present in the RAP α-helix are also considered (rigid_{—}5).

All the rigid polypeptides designed by the inventors are shown in Table 8.

TABLE 8
Proposed rigid polypeptides for the binding of LA module. TPSA is the calculated Topological
Polar Surface Area, which gives insights on the polarity of a polypeptide. Rigid 1 is SEQ ID NO: 12;
rigid 2 is SEQ ID NO: 13; rigid 3 is SEQ ID NO: 14; rigid 4 is SEQ ID NO: 15; rigid 5 is SEQ ID NO:
16.

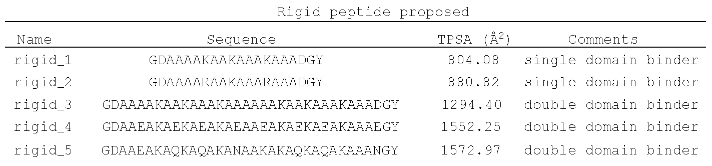

The structures of all the rigid polypeptides designed by the inventors were predicted using the ab-initio method described above [36]. The ab-initio model predicts α-helix structures for all the proposed polypeptides. Two trends were observed (considering polypeptides of same length):
The fewer the number of alanines, the lower the chances that the polypeptide has an α-helix structure.
The more alanine residues, the more probable the polypeptide to be insoluble.

Fluorescently-Labelled Peptide in Vitro BBB Model Crossing Test

Materials & Equipment

Materials
Ringer-Hepes buffer (RH buffer):

NaCl 150 mM; KCL 5,2 mM; CaCl₂ 2.2 mM; MgCl₂ 0.2 mM; NaHCO₃ 6 mM; Hepes 5mM; Glucose 2.8 mM. Adjust to pH 7.2-7.4 and filter through a 0.22 μm filter pore size.
-DMEM/F 12
Lucifer Yellow-CH, Sigma, ref L0259

Equipment
Cell culture incubator (37° C., humidified atmosphere, 95% air and 5% CO2)
Sterile cell culture cabinet
Millicell 24-well Receiver Tray with Lid, Millipore ref PSMW010R5.
Corning 96-well Solid Black Flat Bottom Polystyrene TC-Treated Microplates.
Water bath, 37° C.
Aspiration system
Automatic micropipettor
Fluorimeter

Establishment of an in Vitro BBB Model

A co-culture method is performed as illustrated in FIG. 22. Mouse primary cultures of mixed glial cells are seeded on a 24-well plate and bovine brain endothelial cells are cultured on collagen-coated inserts in another plate. After three days, the inserts are moved into astrocyte-containing plates and cultured for a further three days. To assess the integrity of the in vitro BBB, trans-endothelial electrical resistance (TEER) is measured on the day of the experiment. TEER values higher than 200Ω/cm² are considered acceptable.

Filter Test

The following procedure is followed to test whether the membrane can prevent peptides from crossing from the upper compartment to the lower compartment. Peptides are applied at 20 μg/ml of fluorophore in the luminal side of the inserts and placed in wells filled with Ringer-Hepes buffer (the composition of Ringer-Hepes buffer is described above). After 1 h incubation, samples from the upper and lower compartments are collected and measured using a fluorimeter. Equal peptide concentrations in both compartments indicates that diffusion of the peptides is not restricted by the membrane.

Crossing Test Procedure

Each of the tested peptide solutions are prepared in DMEM/F12 media at a final fluorophore concentration of 20 μg/ml (taking into account that each condition will be studied in triplicate). A fluorescently-labeled scrambled peptide sample is used as a negative control. 20 μM Lucifer Yellow (LY) is added to each sample. LY is a small hydrophilic molecule which presents low cerebral penetration, and so its endothelial permeability coefficient reveals the integrity of the endothelial cell monolayer. It is important to choose a molecular tracer that is compatible with the Em/Ex spectra of the fluorescent label carried by the testing peptides to prevent FRET-like events.

Inserts are moved to a new 24-well plate, with 0.8 ml of media in each well, and warmed at 37° C. 400 μl of pre-warmed sample solutions are added to each insert. An aliquot of each solution is collected (t=0 up), stored at 4° C., and protected from light exposure as well as media (t=0 down). Up and down solutions are collected (t=60 up and t=60 down) after 60 min incubation at 37° C. and 5% CO₂.

All samples (t=0 up, t=0 down, t=60 up and t=60 down) from each condition (testing peptides and negative control) are plated in black 96-well microplates as well as the corresponding standard curves. Fluorescence is measured using a multi-well plate reader.

Mass balances of peptides are then performed to check for possible adsorption or accumulation phenomena. The mass balance value gives the percentage of compound recovered at the end of the experiment, and is calculated as shown below:

$$MB ( \% ) = \frac{\begin{matrix}Peptide amount ( Luminal , t 60 ) + \\ Peptide amount ( Abluminal , t 60 )\end{matrix}}{Peptide amount ( Lum , t 0 )}$$

Permeability Calculation

The endothelial permeability coefficient values for LY and the tested peptide are also determined. The clearance principle is used to obtain a concentration-independent transport parameter. The increment in cleared volume between the incubation times is calculated by dividing the amount of transported compound by the donor chamber concentration and calculating the total volume cleared, as shown below:

$$Clearance ( μl ) = \frac{[ C ] a \times Va}{[ C ] l}$$

[C]l represents the initial luminal tracer/peptide concentration, [C]a represents the abluminal tracer/peptide concentration, and Va represents the volume of the abluminal chamber. During the experiment, the clearance volume increases linearly with time. The average cleared volume is plotted against time, and the slope is estimated by linear regression analysis to provide the mean and the Standard Error for the estimate. The slope of the clearance curves for the co-culture is denoted PSt, where PS represents the permeability x surface area product (in microliters per minute). The slope of the clearance curve for the filter only covered with collagen is denoted PSf. The PS value for the endothelial monolayer (PSe) is calculated as shown below:

$$\frac{1}{PSe} = \frac{1}{PSt} - \frac{1}{PSf}$$

The PSe values are divided by the surface area of the filter (0.7 cm² for millicell 24, cell culture insert) to generate the endothelial permeability coefficient (Pe, in centimeters per minute).

LY Pe and test peptide/scramble peptide Pe coefficients are calculated for each sample. If LY Pe coefficient values are between 0.2−0.8×10⁻³ cm.min⁻¹, then the barrier is considered to be intact after the experiment and the permeability values of test peptides and controls are mainly due to trans-cellular flux.

Results

Using this method, fluorescent peptides crossed the model BBB with the following percentages:

Regulon
(SEQ ID NO: 1)
HKKWQFNSPFVPRADEPARKGKVHIPFPLDNITCRVPMAREPTVIHGKRE
VTLHLHPDH.
Percentage BBB crossing: 3.73%
Regulon polypeptides
(SEQ ID NO: 2)
Sequence: PTVIHGKREVTLHL
Percentage BBB crossing: 11.43%
RAP polypeptides
(SEQ ID NO: 4)
Sequence: ELKHFEAKIEKHNHYQKQLE
Percentage BBB crossing: 8.27%
Flexible polypeptides
(SEQ ID NO: 7)
Sequence: TGESNTVRGKRGSYKDENR
Percentage BBB crossing: 8.17%
Rigid polypeptides
(SEQ ID NO: 12)
Sequence: GDAAAAKAAKAAAKAAADGY
Percentage BBB crossing: 7.51%

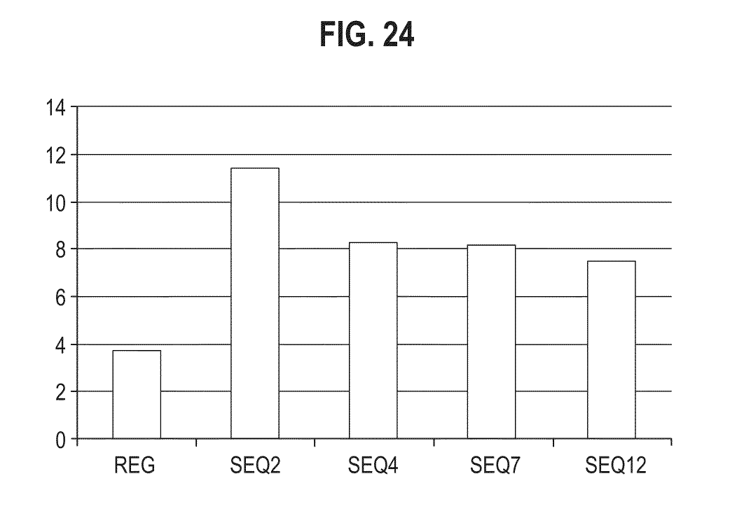

These data are represented graphically in FIG. 24.

### CONCLUSIONS

The inventors have developed polypeptides that undergo receptor-mediated transcytosis of the BBB. Conjugating polypeptides of the invention to an agent makes possible the transport of agents across the BBB into the brain, which would otherwise be excluded by the BBB. Conjugating polypeptides of the invention to a therapeutic agent and/or diagnostic agent, makes possible the transport of therapeutic agents and/or diagnostic agents to the brain, thereby providing new and improved therapeutic and diagnostic possibilities.

| Description of sequence | SEQ ID NO: |
|---|---|
| Regulon | 1 |
| Regulon_construct1 | 2 |
| Regulon_construct4 | 3 |
| RH_construct1 | 4 |
| RH_construct2 | 5 |
| RH_construct3 | 6 |
| Flex_1 | 7 |
| Flex_2 | 8 |
| Flex_3 | 9 |
| Flex_4 | 10 |
| Flex_5 | 11 |
| Rigid_1 | 12 |
| Rigid_2 | 13 |
| Rigid_3 | 14 |
| Rigid_4 | 15 |
| Rigid_5 | 16 |
| Linker | 17 |
| Linker | 18 |
| Linker | 19 |
| Regulon hairpin | 20 |
| Beta strand | 21 |
| Beta strand | 22 |
| Flexible polypeptide consensus sequence | 23 |
| Flexible polypeptide consensus sequence | 24 |
| Flexible polypeptide consensus sequence | 25 |
| Flexible polypeptide consensus sequence | 26 |
| Flexible polypeptide consensus sequence | 27 |
| Flexible polypeptide consensus sequence | 28 |
| Flexible polypeptide consensus sequence | 29 |
| Flexible polypeptide consensus sequence | 30 |
| Flexible polypeptide consensus sequence | 31 |
| Flexible polypeptide consensus sequence | 32 |
| Flexible polypeptide consensus sequence | 33 |
| Flexible polypeptide consensus sequence | 34 |
| Flexible polypeptide consensus sequence | 35 |
| Flexible polypeptide consensus sequence | 36 |
| Flexible polypeptide consensus sequence | 37 |
| Flexible polypeptide consensus sequence | 38 |
| Flexible polypeptide consensus sequence | 39 |
| Flexible polypeptide consensus sequence | 40 |
| Flexible polypeptide consensus sequence | 41 |
| Flexible polypeptide consensus sequence | 42 |
| Flexible polypeptide consensus sequence | 43 |
| Flexible polypeptide consensus sequence | 44 |
| Flexible polypeptide consensus sequence | 45 |
| Flexible polypeptide consensus sequence | 46 |
| Flexible polypeptide consensus sequence | 47 |
| Receptor associated protein | 48 |

### REFERENCES

1 Demeule M., Régina A., Ché C., Poirier J., Nguyen T., Gabathuler R., Castaigne J-P, Béliveau R., Journal of Pharmacology andl Experimental Therapeutics (2007) 324, 1046-1072.

2 Demeule M. Currie J-C, Bertrand Y., Che C., Nguyen T., Regina A., Gabathuler R., Castaigne J-P, Beliveau R., Journal of Neurochemistry (2008) 106, 1534-1544.

3 Brown M. S., Goldstein, J. L., Science (1986) 232, 34-47.

4 Lillis A. P., Van Duyn L. B., Murphy-Ullrich J. E., Strickland D. K., Physiol. Rev., (2008) Vol. 88., 887-918.

5 Russell D. W., Brown M. S., Goldstain J. L., J Biol. Chem (1989) 264, 21682-21688.

6 Cecchelli et al, Adv Drug Deliv Rev. 1999 April 5;36(2-3):165-178

7 U.S. Pat. No. 5,707,829

8 Current Protocols in Molecular Biology (F. M. Ausubel et al. eds., 1987) Supplement 30.

9 Ausubel et al. (eds) (2002) Short protocols in molecular biology, 5th edition (Current Protocols).

10 Gennaro (2000) Remington: The Science and Practice of Pharmacy. 20th edition, ISBN: 0683306472.

11 Methods In Enzymology (S. Colowick and N. Kaplan, eds., Academic Press, Inc.)

12 Handbook of Experimental Immunology, Vols. I-IV (D. M. Weir and C. C. Blackwell, eds, 1986, Blackwell Scientific Publications)

13 Sambrook et al. (2001) Molecular Cloning: A Laboratory Manual, 3rd edition (Cold Spring Harbor Laboratory Press).

14 Handbook of Surface and Colloidal Chemistry (Birdi, K. S. ed., CRC Press, 1997)

15 Ausubel et al. (eds) (2002) Short protocols in molecular biology, 5th edition (Current Protocols).

16 Molecular Biology Techniques: An Intensive Laboratory Course, (Ream et al., eds., 1998, Academic Press)

17 PCR (Introduction to Biotechniques Series), 2nd ed. (Newton & Graham eds., 1997, Springer Verlag)

18 Guttman M., Prieto J. H., Handel T. M., Domaille P. J., Komives E. A., J. Mol. Biol. (2010) 398, 306-319.

19 Guttman M., Prieto J. H., Croy J. E., and Komives E. A., Biochemistry (2010) 49, 1207-1216.

20 Croy J. E., Brandon T., Komives E. A., Biochemistry (2004) 43, 7328-7335.

21 Fisher C., Beglova N., Blacklow S. C., Mol. Cell. (2006) 22, 227-283.

22 Rudenko G., Henry L., Henderson K., Ichtchenko K., Brown M. S., Goldstein J. L., Deisenhofer J., Science (2002)298 (5602): 2353-8.

23 Verdaguer N., Fita I., Reithmayer M., Moser R., Blaas D., Nature Struct. Mol. Biol. (2004) Vol. 11, Number 5, 429.

24 Lee C J, De Biasio A and Beglova N., Structure (2010) 18, 366-376.

25 Beglov D., Lee C-J, De Biasio A., Kozakov D., Brenke R., Vajda S., Beglova N., Proteins (2009) 77 (4), 940-949.

26 Pennings M T, van Lummel M, Derksen R H, Urbanus R T, Romijn R A, Lenting P J, de Groot P G. J. Thromb. Haemost. 2006; 4(8):1680-1690

27 Yasui N., Nogi T., Takagi J., Structure (2010) 10, 18(3): 320-331.

28 Lee C J, De Biasio A and Beglova N., Structure (2010) 18, 366-376 .

29 Van Lummel M., Pennings M. T. T, Derksen¶ R. H. W. M., Urbanus R. T., Lutters B. C. H., Kaldenhoven N., de Groot P. G., J. Biological Chem. (2005) vol. 280, 44, 36729-36736.

30 Jensen G. A., Andersen O. M., Bonvin A. M., Bjerrum-Bohr I, Etzerodt M., Thogersen H. C., O'Shea C., Pulsen F. M., Kragelund B. B., J. Mol. Biol. (2006) 362, 700-716.

31 Beglova N., Jeon H., Fisher C., Blacklow S. C., Mol Cell. (2004) 22, 16(2): 281-292.

32 Fass D., Blacklow S., Kim P. S. Berger J. M., Nature (1997), 388, 691-693

33 Andersen O. M., Christensen L. L., Christensen P. A., Sorensen E. S., Jacobsen C., Moestrup S. K., Etzerodt M., Thogersen H. C. J Biol. Chem. (2000) 275, 21017-21024.

34 Blacklow S.C., Curr. Opin. Struct. Biol. (2007) 17 (4), 419-426.

35 Cai J., Ou R., Xu Y-S, Yang L., Linc Z. , Shu M., J Pept. Sci. 2010, 16: 627-632.

36 Maupetit J., Derreumaux P., Tuffery P., J Comput Chem. 2010, 31(4): 726-38.

37 Maupetit J., Tuffery P., Derreumaux P., Proteins. 2007, 69, 394-408.

38 Kaur H., Garg A., Raghava G. P.., Protein Pept Lett. 2007, 14, 626-31.

39 Halgren T. A., J. Comp. Chem.; 1996, 490-519.

40 Costantini S., Colonna G., Facchiano A. M., Biochemical and Biophysical Research Communications (2006) 342, 441-451.

41 Chou P. Y., Fasman G. D., Biochemistry (1974) 13, 222-245.

42 Chou P. Y., Prediction of Protein Structure and the Principles of Protein Conformation, Plenum Press, New York: Fasman G D, 1989, p. 549

43 Alias M., Ayuso-Tejeidor S., Fernandez-Recio J., Cativiela C., Sancho J., Org. Biomol. Chem. (2010) 8, 788-792.

44 Chakrabartty A., Kortemme Y., Padmanabhan S., Baldwin R. L., Biochemistry (1993) 32, 5560-5565.

45 A. Siflinger-Birnboim, P. J. Del Becchio, J. A. Cooper, F. A. Blumenstock, J. N. Shepard, A. B. Malik, Molecular sieving characteristics of the cultured endothelial monolayer, J. Cell. Physiol. 132 (1987) 111-117.

1. A polypeptide for crossing the blood brain barrier (BBB), wherein the polypeptide is:
(a) a regulon polypeptide less than 59 amino acids in length, comprising 7 or more consecutive amino acids of SEQ ID NO: 2, and comprising K48 and R49 (numbered relative to SEQ ID NO: 1);
(b) a RAP polypeptide less than 100 amino acids in length comprising at least 20 consecutive amino acids from SEQ ID NO: 4;
(c) a flexible polypeptide less than 100 amino acids in length, comprising a flexible loop and wherein the polypeptide comprises the sequence:
X₁ X₂ E X₃ X₄ X₅ X₆ R G K R X₇ X₈ X₉ K
D E X₁₀ X₁₁
or
R G K R X₇ X₈ X₉ K D E
wherein X₁=A, F, S or T; X₂=G, K, R or S; X₃=S or T;X₄=N or S; X₅=A, I or T; X₆=I, T or V; X₇=D, E or G; X₈=T or Y; X₉=F, T or Y; X₁₀=G or N; X₁₁=K or R; or
(d) a rigid polypeptide less than 100 amino acids in length, comprising an alpha helix and comprising the consensus sequence:
(K/R) A (A/E/Q) K A (A/E/Q) A (K/R), optionally
G D (A/E)_{α} (K/R) A (A/E/Q) K A (A/E/Q) A (K/R) A X₆₂ G Y
wherein preferably, ₆₀ is 1-10, and ₆₂ is 1-25.

2. A regulon polypeptide according to claim 1(a), comprising:
(a) (i) T43, V44, 145, H46, G47 and/or (ii) E50, V51, T52, L53 and H54 (numbered relative to SEQ ID NO: 1); and optionally
(b) P43 and L55 (numbered relative to SEQ ID NO: 1); and optionally
(c) (i) P37, M38, A39, R40, E41 and/or (ii) H56, P57, D58, and H59, (numbered relative to SEQ ID NO: 1).

3. The polypeptide according to claim 1, wherein the polypeptide:
(a) is a regulon polypeptide that comprises or consists of SEQ ID NO: 2 or SEQ ID NO: 3;
(b) is a RAP polypeptide that comprises or consists of SEQ ID NO: 4, SEQ ID NO: 5 or SEQ ID NO: 6;
(c) is a flexible polypeptide that comprises or consists of SEQ ID NO: 7, SEQ ID NO: 8, SEQ ID NO: 9, SEQ ID NO: 10, or SEQ ID NO: 11;
(d) is a rigid polypeptide that comprises or consists of SEQ ID NO: 12, SEQ ID NO: 13, SEQ ID NO: 14, SEQ ID NO: 15, or SEQ ID NO: 16.

4. The polypeptide according to claim 1, wherein the polypeptide is produced recombinantly.

5. The polypeptide according to claim 1, wherein the polypeptide is produced by chemical synthesis.

6. A conjugate for transporting an agent across the blood brain barrier (BBB), comprising:
(a) a peptide according to claim 1; and
(b) an agent,
wherein the conjugate is able to cross the BBB.

7. The conjugate according to claim 6, wherein the agent is a drug, a polypeptide, an enzyme, an antibiotic, an anti-cancer agent, a radioactive agent, an antibody, a cellular toxin, a detectable label or an anti-angiogenic compound.

8. The conjugate according to claim 6, wherein the agent is a therapeutic agent.

9. The conjugate according to claim 8, wherein the agent is a small molecule drug.

10. The conjugate according to claim 6, wherein the agent is a diagnostic agent, optionally wherein the diagnostic agent is a dye, a chemi-luminescent dye, radioimaging agent, metal chelate complex, fluorescent label, enzyme-substrate label, an antibody or an antibody fragment thereof.

11. The conjugate according to claim 6, wherein the polypeptide is conjugated to the agent via a linker.

12. The conjugate according to claim 6, wherein the polypeptide is conjugated directly to the agent.

13. The conjugate according to claim 6, further comprising a nanoparticle.

14. The conjugate according to claim 11, wherein the agent is releasable from the polypeptide after transport across the BBB.

15-19. (canceled)

20. An isolated polynucleotide encoding the polypeptide according to claim 1.

21. A pharmaceutical composition, comprising:
a pharmaceutically acceptable carrier; and
at least one of:
(a) the polypeptide according to claim 1; and
(b) a conjugate comprising the polypeptide according to claim 1 and an agent, wherein the conjugate is able to cross the blood brain barrier.

22. A method of treating a neurological disease, comprising the step of:
administering to a patient a composition, wherein the composition is transported across the blood brain barrier for treatment of the neurological disease, and wherein the composition comprises at least one of:
(a) the polypeptide according to claim 1;
(b) a conjugate comprising the polypeptide according to claim 1 and an agent, wherein the conjugate is able to cross the blood brain barrier; and
(c) a pharmaceutical composition comprising a pharmaceutically acceptable carrier and (a) and/or (b).

23. The method of claim 22, wherein the neurological disease is at least one of a brain tumor, brain metastasis, schizophrenia, epilepsy, Alzheimer's disease, Parkinson's disease, Huntington's disease, stroke, and/or a disease associated with malfunction of the BBB.

24. A method of diagnosis of a neurological disease, comprising the step of:
administering to a patient a diagnostic composition, wherein the diagnostic composition is transported across the blood brain barrier for diagnosis of the neurological disease, and wherein the composition comprises at least one of:
(a) the polypeptide according to claim 1;
(b) a conjugate comprising the polypeptide according to claim 1 and an agent, wherein the conjugate is able to cross the blood brain barrier; and
(c) a pharmaceutical composition comprising a pharmaceutically acceptable carrier and (a) and/or (b).

25. The method of claim 24, wherein the neurological disease is at least one of a brain tumor, brain metastasis, schizophrenia, epilepsy, Alzheimer's disease, Parkinson's disease, Huntington's disease, stroke, and/or a disease associated with malfunction of the BBB.
